(19) United States
(12) Patent Application Publication
(10) Pub. No.: US 2024/0270720 A1
(43) Pub. Date: Aug. 15, 2024

(54) INDAZOLE YL BENZIMIDAZOLE DERIVATIVE OR PHARMACEUTICALLY ACCEPTABLE SALT THEREOF, AND USE THEREOF

(71) Applicant: INDUSTRY-UNIVERSITY COOPERATION FOUNDATION HANYANG UNIVERSITY ERICA CAMPUS, Ansan-si Gyeonggi-do (KR)

(72) Inventors: Jung Mi HAH, Seoul (KR); Da Seul IM, Ansan-si Gyeonggi-do (KR)

(21) Appl. No.: 18/576,535

(22) PCT Filed: Jul. 11, 2022
(86) PCT No.: PCT/KR22/10018

§ 371 Date: Jan. 4, 2024

(30) Foreign Application Priority Data
Jul. 12, 2021 (KR) 10-2021-0091215
Jul. 5, 2022 (KR) 10-2022-0082415

(51) Int. Cl. C07D 403/04; A61K 31/4184; A61K 31/422; A61K 31/454; A61K 31/496; A61K 31/5377; C07D 401/14; C07D 403/14; C07D 413/14

(52) U.S. Cl. CPC C07D 403/04; A61K 31/4184; A61K 31/422; A61K 31/454; A61K 31/496; A61K 31/5377; C07D 401/14; C07D 403/14; C07D 413/14

(57) ABSTRACT

Disclosed herein are an indazole yl benzimidazole derivative or a pharmaceutically acceptable salt thereof, and a composition for preventing, relieving or treating a protein kinase-related disease, including the derivative or the salt as an active ingredient, and the like. Further disclosed herein is a method for preventing or treating cancers, inflammatory diseases, or osteoporosis including administering the indazole yl benzimidazole derivative. The indazole yl benzimidazole derivative selectively inhibits Fms-like tyrosine kinase 3 (FLT3) when administered to a subject, and thus may be utilized for preventing, relieving or treating cancers including leukemia, inflammatory diseases including arthritis, or osteoporosis.

### TECHNICAL FIELD

The present disclosure relates to an indazole yl benzimidazole derivative or a pharmaceutically acceptable salt thereof, a use thereof, and a preparation method thereof.

### BACKGROUND ART

Acute myeloid leukemia (AML), one of infamous types of leukemia, is characterized by abnormal proliferation and accumulation of immature cells in the bone marrow and peripheral tissues. The AML leads to a lack of normal hematopoietic cells, resulting in symptoms such as serious infections, fatigue, anemia, breathing difficulties, and bleeding caused by poorly differentiated progenitor cells. According to 2020 AML estimates of the American Cancer Society, the number of newly diagnosed AML patients and the number of patients who died from AML in the United States are high at 19,940 and 11,180, respectively. The incidence of AML generally increases with age, and the overall 5-year survival rate for AML patients is less than 50%. According to recent genome sequencing analysis, mutations in FLT3 are frequently found in AML patients, and FLT3 has been reported as a potential therapeutic target for AML.

Fms-like tyrosine kinase 3 (FLT3), classified as a type of trans-membrane receptor tyrosine kinase, is expressed in lympho-hematopoietic cells. When a Fms-related tyrosine kinase 3 (FLT3) ligand binds to receptor tyrosine kinase (RTK), FLT3 is activated by dimerization and autophosphorylation of a kinase domain, which activates a downstream signal pathway including Janus kinase/signal transducer and activator of transcription (JAK/STAT), Ras/mitogen activated protein kinase (RAS/MAPK), and phosphatidylinositol-3-kinase/AKT/mammalian target of rapamycin (PI3K/AKT/mTOR) pathways, which mediate the immune response, proliferation, and survival of hematopoietic stem cells and progenitor cells.

However, the mutations in FLT3 may cause autophosphorylation and activation without FLT3 ligand binding. FLT3 internal tandem duplication (FLT3-ITD) mutations occur in the juxtamembrane domain (JMD) in the form of sequence duplications, are found in 20-30% of AML patients, and are mainly associated with poor prognosis in AML. FLT3-ITD insertion mutations are frequently observed between Tyr591 and Val592 or Phe594 and Arg595. FLT3 point mutations in the tyrosine kinase domain (TKD) are present in 5% of AML patients, and the most common Asp835 mutation is considered as a part of an AML drug resistance mechanism.

All known FLT3 inhibitors may be classified as type I or type II depending on a binding type. The type I inhibitors are competitive inhibitors capable of binding to an active form (DFG-in form) of FLT3. Sunitinib, midostaurin, lestaurtinib, crenolanib, and gilteritinib have been reported as type I inhibitors, which are closely bound with the active form of FLT3. However, the type I inhibitors lack selectivity for FLT3 and show strong affinity for other kinases due to the high similarity of ATP binding sites. The type II inhibitors interact with a DFG-out form, i.e., the inactive form of FLT3, and also bind to an additional hydrophobic site adjacent to an ATP binding pocket. The type II inhibitors generally have higher selectivity for target kinases because the hydrophobic pocket is a less conserved region compared to the ATP binding site. Sorafenib and quizartinib (VANFLYTA®, Japan) have been reported as the type II inhibitors. Among the above-mentioned FLT3 inhibitors, only two molecules, including midostaurin (Rydapt®) and gilteritinib (Xospata®), received FDA approval for the treatment of FLT3-mutant AML in 2017 and 2018, respectively, and quizartinib received regulatory approval in Japan for FLT3-ITD positive patients in 2019. However, sorafenib (IC₅₀>2000 nM), tandutinib (IC₅₀>10000 nM) and quizartinib (IC₅₀>100 nM) did not show strong inhibitory activity against FLT3-D835Y.

Accordingly, the present inventors analyzed molecular docking and identified that the core structure containing benzimidazole plays a key role in interacting with the Phe691 residue of FLT3 kinase through π-π interaction, and induced the activity enhancement using an indazole fragment as a hinge binder for binding the ATP binding pocket. Thereafter, the present inventors synthesized a novel indazole yl benzimidazole derivative including substituents suitable for a hydrophobic pocket adjacent to a FLT3 active site by performing additional optimizations to improve the processing characteristics and flexibility of the structure, identified the selective inhibitory activity of the derivative against FLT3 kinase, and then completed the present disclosure.

### DISCLOSURE OF THE INVENTION

### Technical Goals

An aspect of the present disclosure is to provide a novel indazole yl benzimidazole derivative or a pharmaceutically acceptable salt thereof.

Another aspect of the present disclosure is to provide a composition for inhibiting Fms-like tyrosine kinase 3 (FLT3) including the indazole yl benzimidazole derivative or the pharmaceutically acceptable salt thereof.

Yet another aspect of the present disclosure is to provide a composition for preventing, relieving, or treating cancers, inflammatory diseases, or osteoporosis including the indazole yl benzimidazole derivative or the pharmaceutically acceptable salt thereof.

Yet another aspect of the present disclosure is to provide a preparation method of the indazole yl benzimidazole derivative or the pharmaceutically acceptable salt thereof.

However, technical goals to be achieved are not limited to those described above, and other goals not mentioned above are clearly understood by one of ordinary skill in the art from the following description.

### Technical Solutions

An aspect of the present disclosure provides an indazole yl benzimidazole derivative represented by Chemical Formula 1 below or a pharmaceutically acceptable salt thereof.

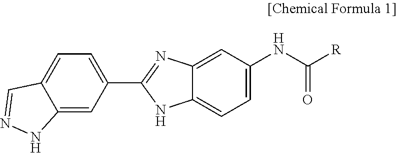

In Chemical Formula 1
a R group is any one selected from the group consisting of a C₃-C₁₀ aryl group, a C₃-C₁₀ heteroaryl group, a C₅-C₁₀ arylvinyl group, a C₅-C₁₀ heteroarylvinyl group, a C₃-C₁₀ arylamino group, and a C₃-C₁₀ heteroarylamino group,
the R group may be substituted with at least one selected from the group consisting of a halogen group, a trifluoromethyl group (CF₃), a C₁-C₆ acyclic or cyclic alkyl group, a C1-C6 alkoxy group, a C₃-C₁₀ heterocyclo group, a C₃-C₁₀ aryl group, a C₃-C₁₀ heteroaryl group, a C₃-C₁₀ heterocycloalkyl group, a C₃-C₁₀ heterocycloamino group, and a C₃-C₁₀ heterocycloether group (wherein, one or more hydrogens of the heterocyclo group, aryl group, heteroaryl group, heterocycloalkyl group, heterocycloamino group, or heterocycloether group are each unsubstituted or substituted with at least one selected from the group consisting of a halogen group, a hydroxy group, a cyano group, a nitro group, a C₁-C₆ acyclic or cyclic alkyl group, and a C₁-C₆ alkoxy group).

In an embodiment of the present disclosure, the R group may be any one selected from the group consisting of a phenyl group, a pyrazole group, an isoxazole group, a phenylvinyl group, an isoxazole amino group, and a phenylamino group.

In another embodiment of the present disclosure, the R group may be substituted with at least one selected from the group consisting of a halogen group, a trifluoromethyl group (CF₃), a tert-butyl group, a methoxy group, a piperazinyl group, a morpholinyl group, a pyrrolidinyl group, a phenyl group, an imidazole group, a piperazinyl methyl group, a piperazinylamino group, a piperidinylamino group, and a piperidinyl ether group (wherein, one or more hydrogens of the piperazinyl group, morpholinyl group, pyrrolidinyl group, phenyl group, imidazole group, piperazinylmethyl group, piperazinylamino group, piperidinyl amino group, or piperidinyl ether group may be each unsubstituted or substituted with at least one selected from the group consisting of a halogen group, a hydroxy group, a cyano group, a nitro group, a C₁-C₆ acyclic or cyclic alkyl group, and a C₁-C₆ alkoxy group).

In another embodiment of the present disclosure, the indazole yl benzimidazole derivative represented by Chemical Formula 1 above may be any one selected from the group consisting of the following compounds.
(1)N-(2-(1H-indazol-6-yl)-1H-benzo[di]imidazol-5-yl)-3-(4-methylpiperazin-1-yl)-5-(trifluoromethyl)benzamide (8a);
(2)N-(2-(1H-indazol-6-yl)-1H-benzo[di]imidazol-5-yl)-3-(4-methyl-1H-imidazol-1-yl)-5-(trifluoromethyl)benzamide (8b);
(3)N-(2-(1H-indazol-6-yl)-1H-benzo[di]imidazol-5-yl)-3-(2-methyl-1H-imidazol-1-yl)-5-(trifluoromethyl)benzamide (8c);
(4)N-(2-(1H-indazol-6-yl)-1H-benzo[di]imidazol-5-yl)-3-morpholino-5-(trifluoromethyl)benzamide (8d);
(5)N-(2-(1H-indazol-6-yl)-1H-benzo[di]imidazol-5-yl)-4-(4-methylpiperazin-1-yl)-3-(trifluoromethyl)benzamide (8e);
(6)N-(2-(1H-indazol-6-yl)-1H-benzo[di]imidazol-5-yl)-4-morpholino-3-(trifluoromethyl)benzamide (8f);
(7)N-(2-(1H-indazol-6-yl)-1H-benzo[di]imidazol-5-yl)-1-phenyl-5-(trifluoromethyl)-1H-pyrazole-4-carboxamide (8g);
(8)N-(2-(1H-indazol-6-yl)-1H-benzo[di]imidazol-5-yl)-5-(tert-butyl)isoxazole-3-carboxamide (8h);
(9)N-(2-(1H-indazol-6-yl)-1H-benzo[di]imidazol-5-yl)-4-chloro-3-(trifluoromethyl)benzamide (8i);
(10)N-(2-(1H-indazol-6-yl)-1H-benzo[di]imidazol-5-yl)-3,4-dichlorobenzamide (8j);
(11) (E)-N-(2-(1H-indazol-6-yl)-1H-benzo[di]imidazol-5-yl)-3-(4-methoxyphenyl)acrylamide (8k);
(12) 1-(2-(1H-indazol-6-yl)-1H-benzo[di]imidazol-5-yl)-3-(5-(tert-butyl)isoxazol-3-yl)urea (8l);
(13) 1-(2-(1H-indazol-6-yl)-1H-benzo[di]imidazol-5-yl)-3-(3,4-dichlorophenyl)urea (8m);
(14)N-(2-(1H-indazol-6-yl)-1H-benzo[di]imidazol-5-yl)-3-(3-(dimethylamino)pyrrolidin-1-yl)-5-(trifluoromethyl)benzamide (8n);
(15)N-(2-(1H-indazol-6-yl)-1H-benzo[di]imidazol-5-yl)-3-(3-(diethylamino)pyrrolidin-1-yl)-5-(trifluoromethyl)benzamide (80);
(16)N-(2-(1H-indazol-6-yl)-1H-benzo[di]imidazol-5-yl)-3-(4-cyclopropylpiperazin-1-yl)-5-(trifluoromethyl)benzamide (8p);
(17)N-(2-(1H-indazol-6-yl)-1H-benzo[di]imidazol-5-yl)-3-((4-cyclopropylpiperazin-1-yl)methyl)-5-(trifluoromethyl)benzamide (8q);
(18)N-(2-(1H-indazol-6-yl)-1H-benzo[di]imidazol-5-yl)-3-((4-ethylpiperazin-1-yl)methyl)-5-(trifluoromethyl)benzamide (8r);
(19)N-(2-(1H-indazol-6-yl)-1H-benzo[di]imidazol-5-yl)-3-((1-methylpiperidin-4-yl)amino)-5-(trifluoromethyl)benzamide (8s);
(20)N-(2-(1H-indazol-6-yl)-1H-benzo[di]imidazol-5-yl)-3-((1-ethylpiperidin-4-yl)amino)-5-(trifluoromethyl)benzamide (8t);
(21)N-(2-(1H-indazol-6-yl)-1H-benzo[di]imidazol-5-yl)-3-((1-methylpiperidin-4-yl)oxy)-5-(trifluoromethyl)benzamide (8u);
(22)N-(2-(1H-indazol-6-yl)-1H-benzo[di]imidazol-5-yl)-3-((1-methylpiperidin-3-yl)amino)-5-(trifluoromethyl)benzamide (8v);
(23)N-(2-(1H-indazol-6-yl)-1H-benzo[di]imidazol-5-yl)-4-((4-ethylpiperazin-1-yl)methyl)-3-(trifluoromethyl)benzamide (8w);
(24)N-(2-(1H-indazol-6-yl)-1H-benzo[di]imidazol-5-yl)-4-((1-methylpiperidin-4-yl)oxy)-3-(trifluoromethyl)benzamide (8x);
(25)N-(2-(1H-indazol-6-yl)-1H-benzo[di]imidazol-5-yl)-3-((4-ethyl-3,3-dimethylpiperazine-1-yl)methyl)-5-(trifluoromethyl)benzamide (8y); and
(26)N-(2-(1H-indazol-6-yl)-1H-benzo[di]imidazol-5-yl)-3-(4-ethyl-3,3-dimethylpiperazine-1-yl)-5-(trifluoromethyl)benzamide (8z).

In another embodiment of the present disclosure, the pharmaceutically acceptable salt of the indazole yl benzimidazole derivative may be at least one selected from the group consisting of hydrochloride, bromate, sulfate, phosphate, nitrate, citrate, acetate, lactate, tartrate, maleate, gluconate, succinate, formate, trifluoroacetate, oxalate, fumarate, glutarate, adipate, methanesulfonate, benzenesulfonate, p-toluenesulfonate, camphorsulfonate, sodium salt, potassium salt, lithium salt, calcium salt, and magnesium salt, but is not limited thereto.

Another aspect of the present disclosure provides a composition for inhibiting Fms-like tyrosine kinase 3 (FLT3) including the indazole yl benzimidazole derivative or the pharmaceutically acceptable salt thereof as an active ingredient.

In an embodiment of the present disclosure, the indazole yl benzimidazole derivative or the pharmaceutically acceptable salt thereof may inhibit wild-type FLT3 or FLT3 mutations.

In another embodiment of the present disclosure, the FLT3 mutations may be a FLT3 internal tandem duplication (FLT3-ITD) mutation or a FLT3 point mutation. The FLT3 internal tandem duplication mutation may occur at Tyr591 and Val592 or Phe594 and Arg595, and the FLT3 point mutation may occur within a TKD region. As non-limiting examples, the FLT3 mutations may include FLT3 (ITD)-NPOS, FLT3 (ITD)-W51, FLT3 (D835Y), FLT3 (F594_R595 ins R), FLT3 (F594_R595 ins REY), FLT3 (R595_E596 ins EY), FLT3 (Y591 V592 ins VDFREYEYD), etc., but is not limited thereto.

Yet another aspect of the present disclosure provides a pharmaceutical composition for preventing or treating cancers, inflammatory diseases, or osteoporosis including the indazole yl benzimidazole derivative or the pharmaceutically acceptable salt thereof as an active ingredient.

Yet another aspect of the present disclosure provides a method for preventing or treating cancers, inflammatory diseases, or osteoporosis including administering the indazole yl benzimidazole derivative or the pharmaceutically acceptable salt thereof to a subject.

Yet another aspect of the present disclosure provides a use of the indazole yl benzimidazole derivative or the pharmaceutically acceptable salt thereof for preparing a drug for the prevention or treatment of cancers, inflammatory diseases, or osteoporosis.

In an embodiment of the present disclosure, the pharmaceutical composition may inhibit FLT3, and specifically, may inhibit wild-type FLT3 or FLT3 mutations.

In another embodiment of the present disclosure, the pharmaceutical composition may further include the indazole yl benzimidazole derivative or the pharmaceutically acceptable salt thereof; and one or more additional ingredients selected from the group consisting of pharmaceutically acceptable carriers, excipients, diluents, stabilizers and preservatives, but the types of additional ingredients are not limited thereto.

In another embodiment of the present disclosure, the pharmaceutical composition may have formulations of powders, granules, tablets, capsules, or injections, but the types of formulations are not limited thereto.

In another embodiment of the present disclosure, the cancer may be at least one selected from the group consisting of leukemia, lymphoma, osteosarcoma, skin cancer, breast cancer, uterine cancer, esophageal cancer, stomach cancer, brain tumor, colon cancer, rectal cancer, colorectal cancer, lung cancer, ovarian cancer, cervical cancer, endometrial cancer, vulvar cancer, kidney cancer, blood cancer, pancreatic cancer, prostate cancer, testicular cancer, laryngeal cancer, head and neck cancer, thyroid cancer, liver cancer, bladder cancer, thymus cancer, urethral cancer, and bronchial cancer, but is not limited thereto. Desirably, the cancer may be leukemia, more desirably acute myelogenous leukemia (AML), chronic myelogenous leukemia (CML), acute lymphocytic leukemia (ALL), chronic lymphocytic leukemia (CLL), acute promyelocytic leukemia (APL), hairy cell leukemia, chronic neutrophilic leukemia (CNL), etc., but is not limited thereto.

In another embodiment of the present disclosure, the inflammatory disease may be at least one selected from the group consisting of arthritis, osteoarthritis, rheumatoid arthritis, psoriatic arthritis, inflammatory arthritis, polyarthritis, glomerulonephritis, inflammatory bowel disease, polymyositis, atopic dermatitis, allergic rhinitis, and asthma, but is not limited thereto.

Yet another aspect of the present disclosure provides a cosmetic composition for preventing or relieving cancers, inflammatory diseases, or osteoporosis including the indazole yl benzimidazole derivative or a cosmetically acceptable salt thereof as an active ingredient.

Yet another aspect of the present disclosure provides a food composition for preventing or relieving cancers, inflammatory diseases, or osteoporosis including the indazole yl benzimidazole derivative or a food acceptable salt thereof as an active ingredient.

Yet another aspect of the present disclosure provides a feed composition for preventing or relieving cancers, inflammatory diseases, or osteoporosis including the indazole yl benzimidazole derivative or a feed acceptable salt thereof as an active ingredient.

Yet another aspect of the present disclosure provides a preparation method of an indazole yl benzimidazole derivative represented by Chemical Formula 1 or a pharmaceutically acceptable salt thereof including:
(1) preparing a compound represented by Chemical Formula 3 from a compound represented by Chemical Formula 2 below;
(2) preparing a compound represented by Chemical Formula 4 from the prepared compound represented by Chemical Formula 3; and
(3) preparing a compound represented by Chemical Formula 1 from the prepared compound represented by Chemical Formula 4.

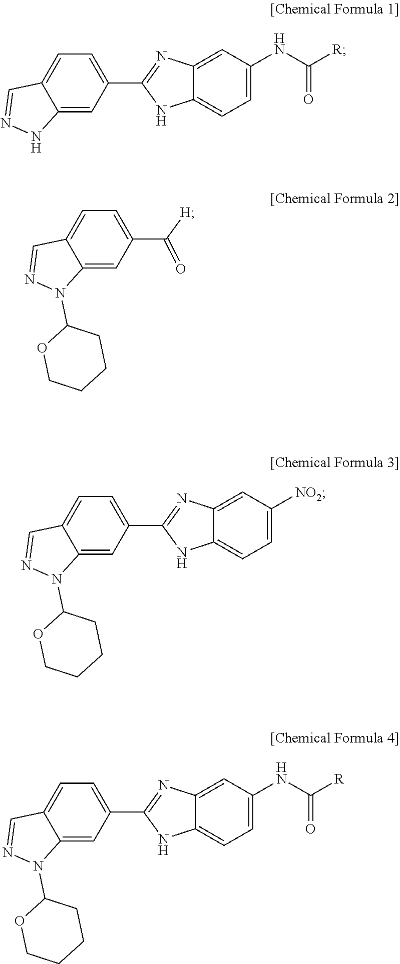

In Chemical Formulas 1 and 4,
a R group is any one selected from the group consisting of a C₃-C₁₀ aryl group, a C₃-C₁₀ heteroaryl group, a C₅-C₁₀ arylvinyl group, a C₅-C₁₀ heteroarylvinyl group, a C₃-C₁₀ arylamino group, and a C₃-C₁₀ heteroarylamino group,
the R group may be substituted with at least one selected from the group consisting of a halogen group, a trifluoromethyl group (CF₃), a C₁-C₆ acyclic or cyclic alkyl group, a C₁-C₆ alkoxy group, a C₃-C₁₀ heterocyclo group, a C₃-C₁₀ aryl group, a C₃-C₁₀ heteroaryl group, a C₃-C₁₀ heterocycloalkyl group, a C₃-C₁₀ heterocycloamino group, and a C₃-C₁₀ heterocycloether group (wherein, one or more hydrogens of the heterocyclo group, aryl group, heteroaryl group, heterocycloalkyl group, heterocycloamino group, or heterocycloether group are each unsubstituted or substituted with at least one selected from the group consisting of a halogen group, a hydroxy group, a cyano group, a nitro group, a C₁-C₆ acyclic or cyclic alkyl group, and a C₁-C₆ alkoxy group).

In an embodiment of the present disclosure, in step 1, a compound represented by Chemical Formula 3 may be prepared by adding 4-nitrobenzene-1,2-diamine, NH₄Cl, and ethanol (EtOH) to the compound represented by Chemical Formula 2.

In another embodiment of the present disclosure, the compound represented by Chemical Formula 2 may be prepared by (i) preparing methyl 1-(tetrahydro-2H-pyran-2-yl)-1H-indazole-6-carboxylate by adding 3,4-dihydro-2H-pyran (DHP) and pyridinium p-toluenesulfonate (PPTS) to methyl 1H-indazole-6-carboxylate, (ii) preparing (1-(tetrahydro-2H-pyran-2-yl)-1H-indazol-6-yl)methanol by adding LiAlH₄, and (iii) oxidizing the prepared compound with at least one selected from the group consisting of Dess-Martin periodinane, pyridinium chlorochromate (PCC), pyridinium dichromate (PDC), Collins-Ratcliff reagent (CrO3·2py), and TPAP(Pr4N⁺RuO4⁻).

In another embodiment of the present disclosure, when the R group is a C₃-C₁₀ aryl group, a C₃-C₁₀ heteroaryl group, a C₅-C₁₀ aryl vinyl group, or a C₅-C₁₀ heteroaryl vinyl group, in step 2, the compound represented by Chemical Formula 4 may be prepared by reducing a nitro group, and then adding RCOOH, 1-ethyl-3-(3-dimethylaminopropyl)carbodiimide (EDC), butoxide (HOBt), and triethanolamine (TEA).

In another embodiment of the present disclosure, when the R group is a C₃-C₁₀ arylamino group, or a C₃-C₁₀ heteroarylamino group, in step 2, the compound represented by Chemical Formula 4 may be prepared by reducing a nitro group, adding 4-nitrophenyl chloroformate and N,N-diisopropylethylamine (DIPEA), and then introducing an R group and heating. At this time, the R group is R′—NH— and may be introduced by adding an amine group, that is, R′—NH₂.

In yet another embodiment of the present disclosure, the reduction may be performed by treating the nitro group with hydrogen (H₂) and palladium/carbon (Pd/C) to be reduced into an amino group.

In another embodiment of the present disclosure, in step 3, the compound represented by Chemical Formula 1 may be prepared by deprotecting the compound represented by Chemical Formula 4 under acidic conditions.

In yet another embodiment of the present disclosure, in step 3, for acidic conditions, trifluoroacetic acid (TFA) or hydrochloric acid (HCl) may be added to the compound represented by Chemical Formula 4. At this time, in step 3, the solvent may be dichloromethane (CH₂Cl₂) or ethanol (EtOH). Desirably, 20% TFA may be added in a dichloromethane solvent or 5% HCl may be added in an ethanol solvent.

In another embodiment of the present disclosure, an intermediate for preparing the indazole yl benzimidazole derivative represented by Chemical Formula 4 may be at least one selected from the group consisting of the following compounds.
(1) 3-(4-methylpiperazin-1-yl)-N-(2-(1-(tetrahydro-2H-pyran-2-yl)-1H-indazol-6-yl)-1H-benzo[di]imidazol-5-yl)-5-(trifluoromethyl)benzamide (7a);
(2) 3-(4-methyl-1H-imidazol-1-yl)-N-(2-(1-(tetrahydro-2H-pyran-2-yl)-1H-indazol-6-yl)-1H-benzo[di]imidazol-5-yl)-5-(trifluoromethyl)benzamide (7b);
(3) 3-(2-methyl-1H-imidazol-1-yl)-N-(2-(1-(tetrahydro-2H-pyran-2-yl)-1H-indazol-6-yl)-1H-benzo[di]imidazol-5-yl)-5-(trifluoromethyl)benzamide (7c);
(4) 3-morpholino-N-(2-(1-(tetrahydro-2H-pyran-2-yl)-1H-indazol-6-yl)-1H-benzo[di]imidazole-5-yl)-5-(trifluoromethyl)benzamide (7d);
(5) 4-(4-methylpiperazin-1-yl)-N-(2-(1-(tetrahydro-2H-pyran-2-yl)-1H-indazol-6-yl)-1H-benzo[di]imidazol-5-yl)-3-(trifluoromethyl)benzamide (7e);
(6) 4-morpholino-N-(2-(1-(tetrahydro-2H-pyran-2-yl)-1H-indazol-6-yl)-1H-benzo[di]imidazole-5-yl)-3-(trifluoromethyl)benzamide (7f);
(7) 1-phenyl-N-(2-(1-(tetrahydro-2H-pyran-2-yl)-1H-indazol-6-yl)-1H-benzo[di]imidazol-5-yl)-5-(trifluoromethyl)-1H-pyrazole-4-carboxamide (7 g);
(8) 5-(tert-butyl)-N-(2-(1-(tetrahydro-2H-pyran-2-yl)-1H-indazol-6-yl)-1H-benzo[di]imidazole-5-yl)isoxazole-3-carboxamide (7h);
(9) 4-chloro-N-(2-(1-(tetrahydro-2H-pyran-2-yl)-1H-indazol-6-yl)-1H-benzo[di]imidazol-5-yl)-3-(trifluoromethyl)benzamide (7i);
(10) 3,4-dichloro-N-(2-(1-(tetrahydro-2H-pyran-2-yl)-1H-indazol-6-yl)-1H-benzo[di]imidazol-5-yl)benzamide (7j);
(11) (E)-3-(4-methoxyphenyl)-N-(2-(1-(tetrahydro-2H-pyran-2-yl)-1H-indazol-6-yl)-1H-benzo[di]imidazol-5-yl)acrylamide (7k);
(12) 1-(5-(tert-butyl)isoxazol-3-yl)-3-(2-(1-(tetrahydro-2H-pyran-2-yl)-1H-indazol-6-yl)-1H-benzo[di]imidazol-5-yl)urea (7l);
(13) 1-(3,4-dichlorophenyl)-3-(2-(1-(tetrahydro-2H-pyran-2-yl)-1H-indazol-6-yl)-1H-benzo[di]imidazole-5-yl)urea (7m);
(14) 3-(3-(dimethylamino)pyrrolidin-1-yl)-N-(2-(1-(tetrahydro-2H-pyran-2-yl)-1H-indazol-6-yl)-1H-benzo[di]imidazol-5-yl)-5-(trifluoromethyl)benzamide (7n);
(15) 3-(3-(diethylamino)pyrrolidin-1-yl)-N-(2-(1-(tetrahydro-2H-pyran-2-yl)-1H-indazol-6-yl)-1H-benzo[di]imidazol-5-yl)-5-(trifluoromethyl)benzamide (70);
(16) 3-(4-cyclopropylpiperazin-1-yl)-N-(2-(1-(tetrahydro-2H-pyran-2-yl)-1H-indazol-6-yl)-1H-benzo[di]imidazol-5-yl)-5-(trifluoromethyl)benzamide (7p);
(17) 3-((4-cyclopropylpiperazin-1-yl)methyl)-N-(2-(1-(tetrahydro-2H-pyran-2-yl)-1H-indazol-6-yl)-1H-benzo[di]imidazol-5-yl)-5-(trifluoromethyl)benzamide (7q);
(18) 3-((4-ethylpiperazin-1-yl)methyl)-N-(2-(1-(tetrahydro-2H-pyran-2-yl)-TH-indazol-6-yl)-1H-benzo[di]imidazol-5-yl)-5-(trifluoromethyl)benzamide (7r);
(19) 3-((1-methylpiperidin-4-yl)amino)-N-(2-(1-(tetrahydro-2H-pyran-2-yl)-1H-indazol-6-yl)-1H-benzo[di]imidazol-5-yl)-5-(trifluoromethyl)benzamide (7s);
(20) 3-((1-ethylpiperidin-4-yl)amino)-N-(2-(1-(tetrahydro-2H-pyran-2-yl)-1H-indazol-6-yl)-1H-benzo[di]imidazol-5-yl)-5-(trifluoromethyl)benzamide (7t);
(21) 4-((1-methylpiperidin-4-yl)oxy)-N-(2-(1-(tetrahydro-2H-pyran-2-yl)-1H-indazol-6-yl)-1H-benzo[di]imidazol-5-yl)-3-(trifluoromethyl)benzamide (7u);
(22) 3-((1-methylpiperidin-3-yl)amino)-N-(2-(1-(tetrahydro-2H-pyran-2-yl)-1H-indazol-6-yl)-1H-benzo[di]imidazol-5-yl)-5-(trifluoromethyl)benzamide (7v);
(23) 4-((4-ethylpiperazin-1-yl)methyl)-N-(2-(1-(tetrahydro-2H-pyran-2-yl)-TH-indazol-6-yl)-1H-benzo[di]imidazol-5-yl)-3-(trifluoromethyl)benzamide (7w);
(24) 3-((1-methylpiperidin-4-yl)oxy)-N-(2-(1-(tetrahydro-2H-pyran-2-yl)-1H-indazol-6-yl)-1H-benzo[di]imidazol-5-yl)-5-(trifluoromethyl)benzamide (7x);
(25) 3-((4-ethyl-3,3-dimethylpiperazin-1-yl)methyl)-N-(2-(1-(tetrahydro-2H-pyran-2-yl)-1H-indazol-6-yl)-1H-benzo[di]imidazol-5-yl)-5-(trifluoromethyl)benzamide (7y); and
(26) 3-(4-ethyl-3,3-dimethylpiperazin-1-yl)-N-(2-(1-(tetrahydro-2H-pyran-2-yl)-1H-indazol-6-yl)-1H-benzo[di]imidazol-5-yl)-5-(trifluoromethyl)benzamide (7z).

### Effects

The present disclosure relates to an indazole yl benzimidazole derivative or a pharmaceutically acceptable salt thereof, a preparation method thereof, a composition for preventing, relieving or treating a protein kinase-related disease including the derivative or the salt as an active ingredient, and the like. The indazole yl benzimidazole derivative of the present disclosure selectively inhibits Fms-like tyrosine kinase 3 (FLT3) when administered to a subject, and thus may be utilized for preventing, relieving or treating cancers including leukemia, inflammatory diseases including arthritis, or osteoporosis.

The indazole yl benzimidazole derivative according to an example of the present disclosure exhibits excellent inhibitory activity for not only a wild-type FLT3, but also a FLT3 internal tandem duplication (FLT3-ITD) mutation, which is associated with a poor prognosis of acute myeloid leukemia (AML), or FLT3 point mutations, which are frequently observed in AML patients or considered to be a part of a drug resistance mechanism in AML.

The indazole yl benzimidazole derivative according to an example of the present disclosure may specifically inhibit FLT3 among various protein kinases, such as ABL1, AKT1, ALK, Aurora A, AXL, BRAF, BTK, c-Kit, c-MER, c-MET, c-Src, CAMKK1, CDK4/cyclin D1, EGFR, ERK1, FGFR3, FLT3, FMS, FYN, GSK3b, IGF1R, JAK3, KDR/VEGFR2, LCK, LYN, MEK1, PKA, PLK1, RON/MST1R, ROS/ROS1, SYK, TRKC, and TYRO3/SKY That is, the derivative of the present disclosure or the pharmaceutically acceptable salt thereof has excellent selectivity.

The effects of the indazole yl benzimidazole derivative or the pharmaceutically acceptable salt according to an example of the present disclosure, and the composition for preventing, relieving, or treating including the same as an active ingredient are not limited to those mentioned above, and other effects not mentioned will be clearly understood by those skilled in the art from the following description.

### BRIEF DESCRIPTION OF DRAWINGS

FIG. 1 illustrates a docking structure of Compound 8a (Chemical Formula 1-1) in a hydrophobic pocket adjacent to an FLT3 active site (left) and an ATP binding site (right).

FIG. 2 illustrates profile results of Compound 8r (Chemical Formula 1-18, 1 μM) treated with various protein kinases.

FIG. 3 illustrates a docking structure of Compound 8r (Chemical Formula 1-18) in FLT3 (PDB: 4RT7). At this time, a light blue region represents a hydrophobic region around an ethyl substituent.

### BEST MODE FOR CARRYING OUT THE INVENTION

The present disclosure relates to a novel indazole yl benzimidazole derivative or a pharmaceutically acceptable salt thereof, and the derivative is a strong FLT3 inhibitor, has an IC₅₀ in a nanomolar unit for wild-type FLT3 and FLT3 mutants, and exhibits high selectivity for 42 or more protein kinases. FLT3 is associated with various diseases such as cancers including acute myeloid leukemia (AML), inflammatory diseases including arthritis, and osteoporosis, and particularly, FLT3 mutation has a high correlation with drug resistance or prognosis of AML. Accordingly, the present disclosure provides a composition for preventing, relieving or treating cancers, inflammatory diseases, osteoporosis, and the like through structure optimization of the indazole yl benzimidazole derivative.

The structure of the indazole yl benzimidazole derivative according to an example of the present disclosure may be represented by chemical Formula 1 below, and the present disclosure provides the indazole yl benzimidazole derivative or a racemate, an isomer, a solvate or a pharmaceutically acceptable salt thereof.

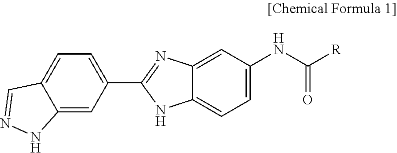

In Chemical Formula 1, a R group is any one selected from the group consisting of a C₃-C₁₀ aryl group, a C₃-C₁₀ heteroaryl group, a C₅-C₁₀ arylvinyl group, a C₅-C₁₀ heteroarylvinyl group, a C₃-C₁₀ arylamino group, and a C₃-C₁₀ heteroarylamino group, and
the R group may be substituted with at least one selected from the group consisting of a halogen group, a trifluoromethyl group (CF₃), a C₁-C₆ acyclic or cyclic alkyl group, a C₁-C₆ alkoxy group, a C₃-C₁₀ heterocyclo group, a C₃-C₁₀ aryl group, a C₃-C₁₀ heteroaryl group, a C₃-C₁₀ heterocycloalkyl group, a C₃-C₁₀ heterocycloamino group, and a C₃-C₁₀ heterocycloether group.

As used in the present disclosure, the term “substitution” refers to a reaction in which atoms or atom groups included in the molecules of the compound are replaced with other atoms or atom groups.

As used in the present disclosure, the term “acyclic” refers to a molecule having a chain structure, and the chain structure is a chemical structure in which carbon atoms are linked to each other in a chain shape, and has a straight chain shape and a branched shape.

As used in the present disclosure, the term “cyclic” refers to a structure in which both ends concatenated in the backbone of an organic compound are linked to each other to form a ring shape.

As used in the present disclosure, the term “acyclic or cyclic alkyl group” means a monovalent linear or branched or cyclic saturated hydrocarbon residue having 1 to 20 carbon atoms and consisting of only carbon and hydrogen atoms. Examples of such an alkyl group include methyl, ethyl, propyl, isopropyl, butyl, isobutyl, 2-butyl, 3-butyl, pentyl, n-hexyl, cyclobutyl, cyclopentyl, cyclohexyl groups, and the like, but are not limited thereto.

As used in the present disclosure, the term “heterocycloalkyl group” usually refers to saturated or unsaturated (but, not aromatic) cyclohydrocarbon, which may optionally be unsubstituted, monosubstituted or polysubstituted, and in its structure, at least one is selected from heteroatoms of N, O or S.

As used in the present disclosure, the term “aryl group” refers to an unsaturated aromatic cyclic compound having 3 to 12 carbon atoms with a single ring (e.g., phenyl) or a plurality of condensed rings (e.g., naphthyl). Examples of such an aryl group include phenyl, naphthyl, and the like, but are not limited thereto.

As used in the present disclosure, the term “heteroaryl group” refers to a single ring or a plurality of condensed rings having at least one heteroatom of N, O or S among atoms constituting the ring. Examples of such a heteroaryl group include a pyridyl group, a pyrimidinyl group, a pyrazinyl group, an oxazolyl group, a furyl group, and the like, but are not limited thereto.

As used in the present disclosure, the term “halogen group” may be fluorine (F), chloride (Cl), bromine (Br), iodine (I), or the like.

As used in the present disclosure, the term “pharmaceutically acceptable salt” means a formulation of a compound that does not cause serious irritation to an organism to which the compound is administered and does not impair the biological activity and physical properties of the compound. The pharmaceutically acceptable salt may be obtained by reacting the compound of the present disclosure with inorganic acids such as hydrochloric acid, bromic acid, sulfuric acid, nitric acid, and phosphoric acid; sulfonic acids such as methanesulfonic acid, ethanesulfonic acid, and p-toluenesulfonic acid; and organic carbonic acids such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, and salicylic acid. In addition, the pharmaceutically acceptable salt may also be obtained by reacting the compound of the present disclosure with bases to form alkali metal salts such as ammonium salt, sodium or potassium salt; salts such as alkaline earth metal salts such as calcium or magnesium salt; salts of organic bases such as dicyclohexylamine, N-methyl-D-glucamine and tris(hydroxymethyl) methylamine; and amino acid salts such as arginine and lysine.

In addition, the indazole yl benzimidazole derivative or the pharmaceutically acceptable salt thereof may include not only pharmaceutically acceptable salts, but also all salts, hydrates and solvates that may be prepared by conventional methods.

In addition, the present disclosure may provide a method for preventing, treating, and/or diagnosing FLT3-related diseases such as cancers, inflammatory diseases, and osteoporosis, including administering the indazole yl benzimidazole derivative or the pharmaceutically acceptable salt thereof to a subject.

As used in the present disclosure, the term “prevention” means all actions that inhibit or delay the occurrence, spread or recurrence of diseases by administering the composition of the present disclosure, and the “treatment” means all actions that improve or beneficially change the symptoms of the diseases by administering the composition of the present disclosure.

As used in the present disclosure, the term “pharmaceutical composition” means a composition prepared for preventing or treating the diseases, and may be formulated and used in various forms according to conventional methods. For example, the pharmaceutical composition may be prepared for oral formulations, such as powders, granules, tablets, capsules, suspensions, emulsions, syrups, and the like, and formulated and used in the form of external preparations, suppositories, and sterile injection solutions.

In the present disclosure, “included as the active ingredient” means that the corresponding ingredient is included in an amount required or sufficient to realize a desired biological effect. In actual application, the amount to be included as an active ingredient is determined as an amount for treating a target disease, and may be determined in consideration of factors that do not cause other toxicity. For example, the amount may vary according to various factors such as a disease or condition to be treated, a type of composition to be administered, the size of a subject, or the severity of a disease or condition. Effective amounts of individual compositions may be determined empirically without undue experimentation by those skilled in the art to which the present disclosure pertains.

In addition, the pharmaceutical composition of the present disclosure may further include one or more pharmaceutically acceptable carriers in addition to the above-described active ingredients according to each formulation.

The pharmaceutically acceptable carrier may be saline, sterile water, a Ringer's solution, buffered saline, a dextrose solution, a maltodextrin solution, glycerol, ethanol, and mixtures of at least one ingredient thereof, and if necessary, may also further include other conventional additives such as antioxidants, buffers, bacteriostats, etc. In addition, the pharmaceutical composition may also be prepared in injectable formulations such as aqueous solutions, suspensions, and emulsions, pills, capsules, granules, or tablets by further adding a diluent, a dispersant, a surfactant, a binder, and a lubricant. Furthermore, the pharmaceutical composition may also be desirably prepared according to each disease or ingredient by a suitable method of the art, or using a method disclosed in Remington's Pharmaceutical Science (Mack Publishing Company, Easton PA).

The composition of the present disclosure may be administered orally or parenterally in a pharmaceutically effective amount according to a desired method. As used in the present disclosure, the term ‘pharmaceutically effective dose’ refers to an amount enough to treat the disease at a reasonable benefit/risk ratio applicable to medical treatment and not to cause side effects. The effective dose level may be determined according to factors including the health condition of a patient, the severity, the activity of a drug, the sensitivity to a drug, an administration method, a time of administration, a route of administration, an emission rate, duration of treatment, and simultaneously used drugs, and other factors well-known in the medical field.

As used in the present disclosure, the term “subject” is any mammal, such as livestock or humans required for prevention, treatment, and/or diagnosis of cancer, but is not limited thereto, and may be desirably humans.

The pharmaceutical composition of the present disclosure may be formulated in various forms for administration to a subject, and a representative formulation for parenteral administration is an injectable formulation, desirably an isotonic aqueous solution or suspension. The injection formulations may be prepared according to techniques known in the art by using suitable dispersing or wetting agents and suspending agents. For example, the injection formulation may be formulated for injection by dissolving each ingredient in saline or buffer. In addition, the formulations for oral administration include, for example, ingestible tablets, buccal tablets, troches, capsules, elixirs, suspensions, syrups, wafers, and the like. In addition to the active ingredients, these formulations may include diluents (e.g., lactose, dextrose, sucrose, mannitol, sorbitol, cellulose and/or glycine) and lubricants (e.g., silica, talc, stearic acid and magnesium or calcium salts thereof and/or or polyethylene glycol). The tablets may include a binder such as magnesium aluminum silicate, starch paste, gelatin, tragacanth, methylcellulose, sodium carboxymethylcellulose and/or polyvinylpyrrolidine, and in some cases, may further include a disintegrant such as starch, agar, alginic acid or sodium salt thereof, an absorbent, a coloring agent, a flavoring agent and/or a sweetening agent. The formulations may be prepared by conventional mixing, granulating or coating methods.

In addition, the pharmaceutical composition of the present disclosure may further include adjuvants such as preservatives, hydrating agents, emulsifying accelerators, and salts or buffers for osmotic pressure control, and other therapeutically useful substances, and may be formulated according to conventional methods.

The pharmaceutical composition according to the present disclosure may be administered through various routes including oral, transdermal, subcutaneous, intravenous or intramuscular route, and the dose of the active ingredient may be appropriately selected according to various factors such as a route of administration, the age, sex, and weight of a patient, and the severity of a patient. In addition, the composition of the present disclosure may be administered in combination with known compounds capable of increasing the desired effect.

As the route of administration of the pharmaceutical composition according to the present disclosure, the pharmaceutical composition may be administered to humans and animals orally or parenterally, such as intravenously, subcutaneously, intranasally or intraperitoneally. The oral administration also includes sublingual application. The parenteral administration includes an injection method such as subcutaneous injection, intramuscular injection and intravenous injection and a dripping method.

In the pharmaceutical composition of the present disclosure, the total effective dose of the indazole yl benzimidazole derivative according to the present disclosure or the pharmaceutically acceptable salt thereof may be administered to a patient in a single dose, or in a multiple dose according to a fractionated treatment protocol administered for a long period of time. In the pharmaceutical composition of the present disclosure, the content of the active ingredient may vary depending on the severity of disease, but generally, the pharmaceutical composition may be repeatedly administered several times a day at an effective dose of 100 μg to 3,000 mg when administered once based on adults. However, in the concentration of the indazole yl benzimidazole derivative or the pharmaceutically acceptable salt thereof, the effective dose to the patient may be determined by considering various factors, such as the age, weight, health condition, and sex of a patient, the severity of a disease, diet and excretion rate as well as a route of administration and the number of treatments of a drug.

In addition, the pharmaceutical composition according to the present disclosure is not particularly limited in formulation, administration route, and administration method thereof as long as the pharmaceutical composition exhibits the effects of the present disclosure. The pharmaceutical composition of the present disclosure may further include, as an active ingredient, known anticancer agents, therapeutic agents for inflammatory diseases, or therapeutic agents for osteoporosis in addition to the indazole yl benzimidazole derivative or the pharmaceutically acceptable salt thereof, and may be used in combination with other treatments known to treat these diseases.

In addition, yet another aspect of the present disclosure provides a cosmetic composition for preventing or relieving cancers, inflammatory diseases, or osteoporosis including the indazole yl benzimidazole derivative or a cosmetically acceptable salt thereof as an active ingredient.

As used in the present disclosure, the term “relieving” means all actions that at least reduce parameters associated with conditions to be treated, for example, the degree of symptoms, or improve or beneficially change the disease.

The cosmetic composition may be prepared in the form of a basic cosmetic composition (lotion, cream, essence, face cleanser such as cleansing foam and cleansing water, packs, and body oils), a colored cosmetic composition (foundation, lipstick, mascara, and makeup base), a hair product composition (shampoo, conditioner, hair conditioner, and hair gel), soap, etc., in addition to dermatologically acceptable excipients.

The excipients may include, for example, skin emollients, skin penetration enhancers, colorants, fragrances, emulsifiers, thickeners and solvents, more specifically starch, glucose, lactose, sucrose, gelatin, malt, rice, wheat flour, chalk, silica gel, sodium stearate, glycerol monostearate, talc, sodium chloride, anhydrous skim milk, glycerol, propylene, glycol, water, ethanol, etc., but are not limited thereto.

In addition, yet another aspect of the present disclosure provides a food composition for preventing or relieving cancers, inflammatory diseases, or osteoporosis including the indazole yl benzimidazole derivative or a food acceptable salt thereof as an active ingredient.

When the food composition uses the indazole yl benzimidazole derivative as an additive of the food composition, the food composition may be added as it is or used with other foods or food ingredients, and may be used appropriately according to conventional methods. In general, when preparing foods or beverages, the composition of the present disclosure is added in an amount of 15 wt % or less, desirably 10 wt % or less, based on the raw material. However, in the case of long-term intake for the purpose of health and hygiene or for the purpose of health control, the amount of the active ingredients may be equal to or greater than the range, and since there is no problem in terms of safety, the active ingredients may be used even in an amount above the range. That is, the mixing amount of the active ingredients may be appropriately determined depending on each purpose of use, such as prevention, health, or treatment.

The formulations of the food composition may be in the form of powders, granules, pills, tablets, capsules, as well as general foods or beverages.

The kind of food is not particularly limited. Examples of the food which may be added with the materials include meat, sausages, bread, chocolate, candies, snacks, confectionery, pizza, ramen, other noodles, gums, dairy products including ice cream, various soups, beverages, tea, drinks, alcohol drinks, vitamin complex, and the like, and include all health functional foods in an accepted meaning.

The food of the present disclosure is able to be prepared by methods which are commonly used in the art and may be prepared by adding raw materials and ingredients which are commonly added in the art in the preparation. Specifically, the food may include proteins, carbohydrates, fats, nutrients, seasonings, and flavoring agents, and examples of the carbohydrates include glucose, fructose, maltose, sucrose, oligosaccharides, dextrin, cyclodextrin, xylitol, sorbitol, erythrol, saccharin, or synthetic flavoring agents, but are not limited thereto.

In addition, yet another aspect of the present disclosure provides a feed composition for preventing or relieving cancers, inflammatory diseases, or osteoporosis including the indazole yl benzimidazole derivative or a feed acceptable salt thereof as an active ingredient.

As used in the present disclosure, the term ‘feed’ may mean any natural or artificial diet, one-meal diet, or ingredients of one-meal diet to be eaten and ingested by livestock or suitable thereto. The feed may include feed additives or supplementary feed.

The type of feed is not particularly limited, and may use feeds commonly used in the art. Non-limiting examples of the feed may include vegetable feeds, such as grains, root fruits, food processing by-products, algae, fibers, pharmaceutical by-products, oils and fats, starches, meals or grain by-products; and animal feeds such as proteins, inorganic materials, oils and fats, minerals, oils and fats, single-cell proteins, animal planktons, foods, etc. These feeds may be used alone or in combination of two or more kinds.

The terms used in the examples are used for the purpose of description only, and should not be construed to be limited. The singular expression includes the plural expression unless the context clearly dictates otherwise. In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which examples pertain. Terms which are defined in a generally used dictionary should be interpreted to have the same meaning as the meaning in the context of the related art, and are not interpreted as ideal or excessively formal meanings unless otherwise defined in the present disclosure.

### MODE FOR CARRYING OUT THE INVENTION

The present disclosure may have various modifications and various Examples, and specific Examples will be hereinafter illustrated in the drawings and described in detail in the detailed description. However, the present disclosure is not limited to specific Embodiments, and it should be understood that the present disclosure covers all the modifications, equivalents and replacements within the idea and technical scope of the present disclosure. In the interest of clarity, not all details of the relevant art are described in detail in the present specification in so much as such details are not necessary to obtain a complete understanding of the present disclosure.

### Example 1. Synthesis of Benzimidazole Derivatives

A general synthetic pathway of benzimidazole derivatives was shown in [Reaction Scheme 1] and [Reaction Scheme 2] below. Methyl 1H-indazole-6-carboxylate (1) was treated with 3,4-dihydro-2H-pyran (DHP) under mildly acidic conditions and subjected to microwave irradiation at 50° C. to obtain Compound (2) protected by introducing a DHP group. Methyl 1-(tetrahydro-2H-pyran-2-yl)-1H-indazole-6-carboxylate (2) was reduced to alcohol (3) using LiAlH₄ and oxidized to an aldehyde compound (4, Chemical Formula 2) using Dess-Martin periodinane. Aldehyde (4) was treated with NH₄Cl and 4-nitrobenzene-1,2-diamine to obtain a benzimidazole compound (5, Chemical Formula 3) as a core intermediate. Next, a nitro group of benzimidazole (5) was reduced to an amino group under H₂. Aniline (6) was coupled with various benzoic acids, and then deprotected under acidic conditions to prepare final benzimidazole derivatives (8a-k, 8n-z, Chemical Formulas 1-1 to 1-11, Chemical Formulas 1-14 to 1-26) (Reaction Scheme 1). In addition, aniline (6) and amine were coupled through urea coupling and deprotected to obtain final benzimidazole derivatives (8l-m, Chemical Formulas 1-12 and 1-13) (Reaction Scheme 2).

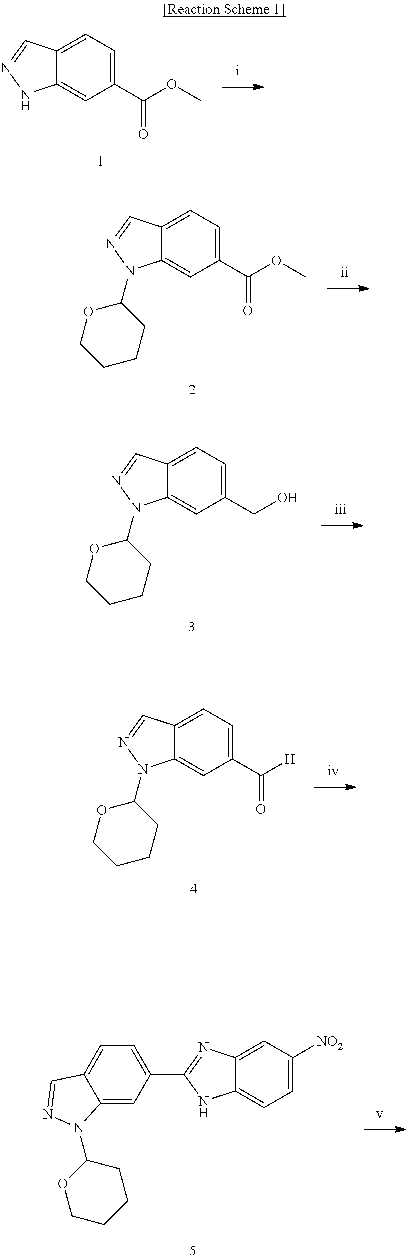 [SMILES: COC(=O)c1ccc2cn[nH]c2c1 ; COC(=O)c1ccc2cnn(C3CCCCO3)c2c1 ; OCc1ccc2cnn(C3CCCCO3)c2c1 ; O=Cc1ccc2cnn(C3CCCCO3)c2c1 ; O=[N+]([O-])c1ccc2[nH]c(-c3ccc4cnn(C5CCCCO5)c4c3)nc2c1]

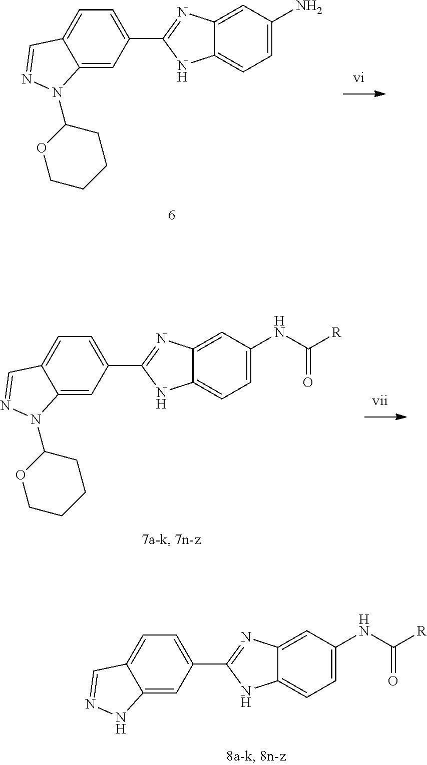

[Reaction Scheme 1] showed the synthesis of a N-(2-(1H-indazol-6-yl)-1H-benzo[di]imidazol-5-yl)benzamide derivative, and the specific conditions were as follows: (i) 3,4-dihydro-2H-pyran, Pyridinium p-toluenesulfonate, μW, 50° C., 5h; (ii) LiAlH₄ in THF, THF, 0° C.; (iii) Dess-Martin periodinane, MC/THF=1:1, rt; (iv) 4-Nitrobenzene-1,2-diamine, NH₄Cl, EtOH, reflux; (v) H₂, Pd/C, EtOH; (vi) RCO₂H, EDC, HOBt, TEA, THF, rt; (vii) 20% TFA, CH₂Cl₂ or 5% HCl in EtOH.

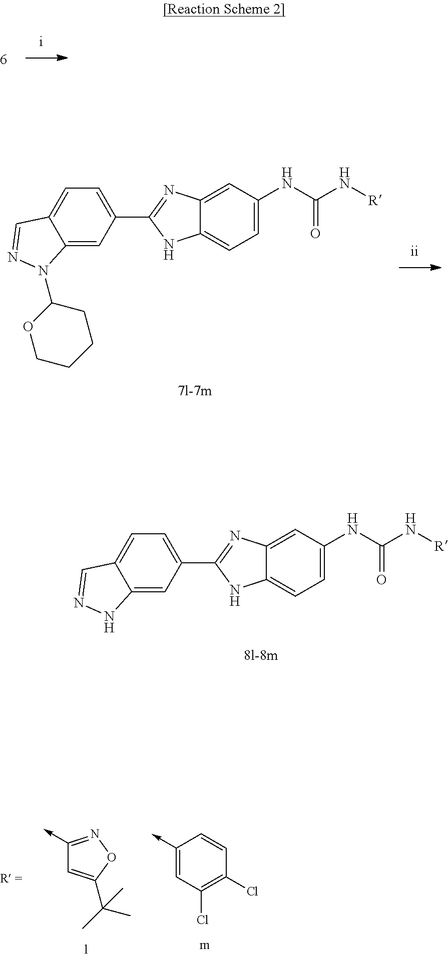

[Reaction Scheme 2] showed the synthesis of a 1-(2-(1H-indazol-6-yl)-1H-benzo[di]imidazol-5-yl)-3-phenylurea derivative, and the specific conditions were as follows: (i) (1) 4-nitrophenyl chloroformate, DIPEA, THF, 0° C.; (2) R′NH₂, THF, 50° C.; (ii) 20% TFA. CH₂Cl₂.

All chemicals were reagent grades and purchased from Aldrich (USA), Alfa aesar (USA), and TCI (Japan). Separation of compounds by column chromatography was performed using silica gel 60 (200-300 mesh ASTM, E. Merck, Germany). The amount of silica gel used was 50 to 200 times the weight filled in the column. Thin layer chromatography (TLC) was performed on a silica gel-coated aluminum sheet (silica gel 60 GF254, E. Merck, Germany) and visualized under ultraviolet (UV) light (254 nm). ¹H NMR and ¹³C NMR spectra were recorded on a Brucker model digital AVANCE III 400M Hz spectrometer at 25° C. using tetramethylsilane (TMS) as an internal standard. High-resolution MS (HR/MS) experiments were performed with a Finnigan LTQ Orbitrap mass spectrometer (Thermo Fisher Scientific Inc, MA, USA) operated in an anion electrospray mode.

### 1.1. Synthesis of methyl 1-(tetrahydro-2H-pyran-2-yl)-1H-indazole-6-carboxylate (2)

A mixture of methyl 1H-indazole-6-carboxylate (1) (1216 mg, 6.90 mmol), DHP (8.28 mmol), and PPTS (0.07 mmol) was reacted at 50° C. for 5 hours under microwave irradiation to obtain methyl 1-(tetrahydro-2H-pyran-2-yl)-1H-indazole-6-carboxylate (2). After completion of the reaction, the reaction mixture was diluted with ethyl acetate and washed with saturated aqueous sodium bicarbonate. The organic layer was dried over Na₂SO₄. The concentrated crude product was purified by flash column chromatography to obtain a desired product as yellow oil (1592.8 mg. 88.7%). ¹H NMR (400 MHz, DMSO-d₆) δ 8.63 (d, J=0.9 Hz, 1H), 8.32 (dd, J=2.3, 1.0 Hz, 1H), 7.84 (dd, J=8.8, 0.9 Hz, 1H), 7.58 (dd, J=8.8, 1.4 Hz, 1H), 5.81 (dd, J=9.5, 2.8 Hz, 1H), 4.00 (dtd, J=8.8, 3.7, 1.6 Hz, 1H), 3.88 (s, 3H), 3.74 (ddd, J=11.5, 8.2, 6.2 Hz, 1H), 2.24-2.14 (m, 1H), 2.09 (dt, J=9.0, 3.4 Hz, 1H), 2.00-1.93 (m, 1H), 1.80-1.70 (m, 1H), 1.65-1.57 (m, 2H). LC/MS (ESI+, m/z) calcd for C₁₃H₁₆N₂O₃Na [M+Na]⁺: 283.1053, found 283.3543.

### 1.2. Synthesis of (1-(tetrahydro-2H-pyran-2-yl)-1H-indazol-6-yl)methanol (3)

A solution of methyl 1-(tetrahydro-2H-pyran-2-yl)-1H-indazole-6-carboxylate (1592.8 mg, 6.12 mmol) in anhydrous THF (30.6 ml) was added dropwise in 2.0 M LAH in THF (3.4 ml). After completion of the reaction, the residue was added with a 1 N aqueous sodium hydroxide solution and extracted with ethyl acetate. The organic layer was washed with saturated saline and dried over anhydrous sodium sulfate. The solvent was distilled off under reduced pressure. The crude compound (1335.1 mg, 93.9%) of (1-(tetrahydro-2H-pyran-2-yl)-1H-indazol-6-yl)methanol was obtained as solid. ¹H NMR (400 MHz, DMSO-d₆) δ 8.05 (d, J=0.5 Hz, 1H), 7.72-7.67 (m, 1H), 7.65 (d, J=0.8 Hz, 1H), 7.16-7.10 (m, 1H), 5.82 (dd, J=9.7, 2.5 Hz, 1H), 5.34 (t, J=5.7 Hz, 1H), 4.65 (d, J J=5.5 Hz, 2H), 3.91-3.85 (m, 1H), 3.73 (ddd, J=11.4, 8.0, 5.9 Hz, 1H), 2.47-2.38 (m, 1H), 2.03 (ddt, J=10.1, 7.9, 4.3 Hz, 1H), 1.99-1.93 (m, 1H), 1.81-1.70 (m, 1H), 1.62-1.54 (m, 2H). LC/MS (ESI+, m/z) calcd for C₁₃H₁₆N₂O₂Na [M+Na]⁺: 255.1104, found 255.0434.

### 1.3. Synthesis of 1-(tetrahydro-2H-pyran-2-yl)-1H-indazole-6-carbaldehyde (4, Chemical Formula 2)

Dess-Martin periodinane (1.437.9 mg, 5.75 mmol) was added to a stirred solution of (1-(tetrahydro-2H-pyran-2-yl)-1H-indazol-6-yl)methanol (1335.1 mg, 5.75 mmol) in DCM (8.3 ml) and THF (8.3 ml) and stirred at room temperature for 16 hours. The mixture was added with DCM and filtered through Celite. The filtrate was evaporated under vacuum to obtain a compound of 1-(tetrahydro-2H-pyran-2-yl)-1H-indazole-6-carbaldehyde (1.4 g). ¹H NMR (400 MHz, DMSO-d₆) δ 10.18-10.15 (m, 1H), 8.41 (d, J=1.0 Hz, 1H), 8.29 (d, J=0.6 Hz, 1H), 7.98 (d, J=8.3 Hz, 1H), 7.70 (dd, J=8.3, 1.2 Hz, 1H), 6.03 (dd, J=9.6, 2.4 Hz, 1H), 3.93 (ddd, J=7.5, 3.5, 1.8 Hz, 1H), 3.81 (ddd, J=11.5, 7.9, 5.7 Hz, 1H), 2.48-2.40 (m, 1H), 2.10-2.01 (m, 2H), 1.84-1.74 (m, 1H), 1.63 (tt, J=8.3, 3.9 Hz, 2H). LC/MS (ESI+, m/z) calcd for C₁₃H₁₄N₂O₂Na [M+Na]⁺: 253.0947, found 253.1530.

### 1.4. Synthesis of 6-(5-nitro-1H-benzo[di]imidazol-2-yl)-1-(tetrahydro-2H-pyran-2-yl)-1H-indazole (5, Chemical Formula 3)

A mixture of 4-nitrobenzene-1,2-diamine (948.6 mg, 6.19 mmol), 1-(tetrahydro-2H-pyran-2-yl)-1H-indazole-6-carbaldehyde (4) (1426.3 mg, 6.19 mmol), NH₄Cl (331.4 mg, 6.19 mmol), and EtOH (61.9 mg) was reacted at 80° C. for 2 hours. After the starting material disappeared, the reaction mixture was diluted with ethyl acetate and washed with saturated aqueous sodium bicarbonate. The organic layer was dried over Na₂SO₄. The concentrated crude product was purified by flash column chromatography to obtain a desired product as yellow solid (548.0 mg. 24.4%). m.p. 142-144° C. ¹H NMR (400 MHz, DMSO-d₆) δ 13.74 (s, 1H), 8.59 (s, 1H), 8.52 (s, 1H), 8.24 (d, J=0.6 Hz, 1H), 8.17 (dd, J=8.9, 2.2 Hz, 1H), 8.05 (dd, J=8.4, 1.3 Hz, 1H), 8.00 (dd, J=8.5, 0.7 Hz, 1H), 7.82 (d, J=8.7 Hz, 1H), 5.99 (dd, J=9.5, 2.2 Hz, 1H), 3.95 (d, J=12.1 Hz, 1H), 3.17 (d, J=5.3 Hz, 2H), 2.14-2.01 (m, 2H), 1.87-1.75 (m, 1H), 1.65 (d, J=3.4 Hz, 2H). ¹³C NMR (101 MHz, DMSO) δ 154.6, 147.4, 143.3, 139.8, 137.5, 135.7, 134.3, 129.5, 127.5, 125.9, 122.3, 120.5, 114.9, 109.8, 84.8, 67.2, 29.4, 25.3, 22.7. LC/MS (ESI+, m/z) calcd for C₁₉H₁₇N₅O₃ [M+H]⁺: 364.1410, found 364.2837.

### 1.5. Synthesis of 2-(1-(tetrahydro-2H-pyran-2-yl)-1H-indazol-6-yl)-1H-benzo[di]imidazol-5-amine (6)

A suspension of Compound 5 (200 mg, 0.551 mmol) and 20 mg of Pd/C (10%) in 5.51 ml of EtOH was stirred under H₂ for 16 hours. After filtration through Celite, the solution was concentrated under reduced pressure to obtain Compound 6 (121.2 mg, 66%). The crude compound of 2-(1-(tetrahydro-2H-pyran-2-yl)-1H-indazol-6-yl)-1H-benzo[di]imidazol-5-amine was used as starting material for the next step without additional purification. m.p. 142-143° C. ¹H NMR (400 MHz, DMSO-d₆) δ 12.47 (s, 1H), 8.40 (s, 1H), 8.16 (s, 1H), 7.95 (dd, J=8.5, 1.0 Hz, 1H), 7.88 (d, J=8.4 Hz, 1H), 7.33 (d, J=6.8 Hz, 1H), 6.73 (s, 1H), 6.57 (d, J=7.9 Hz, 1H), 5.93 (dd, J=9.6, 2.1 Hz, 1H), 5.02 (s, 2H), 3.97 (d, J=11.5 Hz, 1H), 3.88-3.78 (m, 1H), 2.05 (ddd, J=12.5, 9.2, 6.0 Hz, 2H), 1.88-1.74 (m, 1H), 1.65 (dd, J=8.0, 4.2 Hz, 2H), 1.27 (q, J=7.1 Hz, 1H). ¹³C NMR (101 MHz, DMSO) δ 153.2, 144.7, 141.8, 140.1, 134.1, 133.5, 131.2, 130.0, 129.4, 124.6, 121.7, 120.0, 112.7, 112.2, 84.7, 67.2, 29.4, 25.3, 22.8. LC/MS (ESI+, m/z) calcd for C₁₉H₁₉N₅O [M+H]⁺: 334.1668, found 334.3547.

### 1.6. Synthesis of Intermediates (7a-z, Chemical Formula 4) 1.6.1. 3-(4-methylpiperazin-1-yl)-N-(2-(1-(tetrahydro-2H-pyran-2-yl)-1H-indazol-6-yl)-1H-benzo[di]imidazol-5-yl)-5-(trifluoromethyl)benzamide (7a)

EDC (5.87 mg, 0.0306 mmol), HOBt (3.75 mg, 0.0245 mmol), DIPEA (0.0055 ml) and benzoic acid (7.05 mg, 0.0244 mmol) were added to a solution of Compound 6 (6.80 mg, 0.0204 mmol) in THF (0.204 ml). The reactant was stirred at room temperature overnight. After completion of the reaction, the mixture was cooled to ambient temperature and the solvent was removed in vacuo. The reaction mixture was diluted with ethyl acetate and washed with saturated aqueous sodium bicarbonate. The organic layer was dried over Na₂SO₄. The concentrated crude product was purified by flash column chromatography to obtain a desired product as pure solid (4.6 mg). A mixture of 3-(4-methylpiperazin-1-yl)-N-(2-(1-(tetrahydro-2H-pyran-2-yl)-1H-indazol-6-yl)-1H-benzo[di]imidazol-5-yl)-5-(trifluoromethyl)benzamide was separated as pure solid in 37.4% yield. LC/MS (ESI+, m/z) calcd for C₃₂H₃₂F₃N₇O₅ [M+H]⁺: 604.2648, found 604.5891.

### 1.6.2. 3-(4-methyl-1H-imidazol-1-yl)-N-(2-(1-(tetrahydro-2H-pyran-2-yl)-1H-indazol-6-yl)-1H-benzo[di]imidazol-5-yl)-5-(trifluoromethyl)benzamide (7b)

A mixture of 3-(4-methyl-1H-imidazol-1-yl)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7b as pure solid in 8.6% yield. LC/MS (ESI+, m/z) calcd for C₃₂H₂₆F₃N₇O₂ [M+H]⁺: 586.2178, found 586.4406.

### 1.6.3. 3-(2-methyl-1H-imidazol-1-yl)-N-(2-(1-(tetrahydro-2H-pyran-2-yl)-1H-indazol-6-yl)-1H-benzo[di]imidazol-5-yl)-5-(trifluoromethyl)benzamide (7c)

A mixture of 3-(2-methyl-1H-imidazol-1-yl)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7c as pure solid in 43.0% yield. LC/MS (ESI+, m/z) calcd for C₃₁H₂₆F₃N₇O₂ [M+H]⁺: 586.2178, found 586.5126.

### 1.6.4. 3-morpholino-N-(2-(1-(tetrahydro-2H-pyran-2-yl)-1H-indazol-6-yl)-1H-benzo[di]imidazole-5-yl)-5-(trifluoromethyl)benzamide (7d)

A mixture of 3-morpholino-N-(2-(1-(tetrahydro-2H-pyran-2-yl)-1H-indazol-6-yl)-1H-benzo[di]imidazole-5-yl)-5-(trifluoromethyl)benzamide was separated in a similar manner to the synthesis of Compound 7a described above to obtain Compound 7d as pure solid in 36.8% yield. ¹H NMR (400 MHz, DMSO-d₆) δ 10.57 (s, 1H), 8.66 (s, 1H), 8.55 (s, 1H), 8.33 (s, 1H), 7.98 (d, J=8.8 Hz, 1H), 7.92 (dd, J=8.8, 1.4 Hz, 1H), 7.79 (s, 1H), 7.73 (m, 2H), 7.66 (d, J=8.6 Hz, 1H), 7.43 (s, 1H), 5.85 (dd, J=9.5, 2.7 Hz, 1H), 4.05 (d, J=11.2 Hz, 1H), 3.80 (m, 5H), 3.34 (d, J=4.8 Hz, 4H), 2.28-2.19 (m, 1H), 2.17-2.10 (m, 1H), 2.01 (m, 1H), 1.79 (m, 1H), 1.67-1.63 (m, 2H). LC/MS (ESI+, m/z) calcd for C₃₁H₂₉F₃N₆O₃[M+H]⁺: 591.2331, found 591.5899.

### 1.6.5. 4-(4-methylpiperazin-1-yl)-N-(2-(1-(tetrahydro-2H-pyran-2-yl)-1H-indazol-6-yl)-1H-benzo[di]imidazol-5-yl)-3-(trifluoromethyl)benzamide (7e)

A mixture of 4-(4-methylpiperazin-1-yl)-N-(2-(1-(tetrahydro-2H-pyran-2-yl)-1H-indazol-6-yl)-1H-benzo[di]imidazol-5-yl)-3-(trifluoromethyl)benzamide was separated in a similar manner to the synthesis of Compound 7a described above to obtain Compound 7e as pure solid in 49.4% yield. ¹H NMR (400 MHz, DMSO-d₆) δ 13.10 (s, 1H), 10.45 (s, 1H), 8.52 (s, 1H), 8.30-8.20 (m, 4H), 8.04 (dd, J=8.5, 1.2 Hz, 1H), 7.98-7.93 (m, 1H), 7.65 (d, J=8.4 Hz, 2H), 7.54 (d, J=8.6 Hz, 1H), 5.97 (dd, J=9.6, 2.2 Hz, 1H), 3.97 (d, J=11.3 Hz, 1H), 3.88-3.80 (m, 1H), 3.00 (t, J=4.7 Hz, 4H), 2.46 (s, 4H), 2.26 (s, 3H), 2.14-2.03 (m, 2H), 2.02-1.94 (m, 1H), 1.88-1.78 (m, 1H), 1.66 (s, 2H)

### 1.6.6. 4-morpholino-N-(2-(1-(tetrahydro-2H-pyran-2-yl)-1H-indazol-6-yl)-1H-benzo[di]imidazol-5-yl)-3-(trifluoromethyl)benzamide (7f)

A mixture of 4-morpholino-N-(2-(1-(tetrahydro-2H-pyran-2-yl)-1H-indazol-6-yl)-1H-benzo[di]imidazol-5-yl)-3-(trifluoromethyl)benzamide was separated in a similar manner to the synthesis of Compound 7a described above to obtain Compound 7f as pure solid in 12.7% yield. ¹H NMR (400 MHz, DMSO-d₆) δ 13.04 (s, 1H), 10.46 (s, 1H), 8.53 (s, 1H), 8.25 (m, 4H), 8.04 (m, 1H), 7.95 (d, J=8.4 Hz, 1H), 7.69-7.52 (m, 3H), 5.97 (d, J=9.6 Hz, 1H), 3.98 (d, J=11.1 Hz, 1H), 3.84 (dd, J=15.3, 9.2 Hz, 1H), 3.76 (d, J=4.3 Hz, 4H), 3.35 (s, 1H), 2.99 (s, 4H), 2.52 (s, 3H), 2.08 (t, J=13.4 Hz, 2H), 1.81 (d, J=9.0 Hz, 1H), 1.66 (s, 2H). LC/MS (ESI+, m/z) calcd for C₃₁H₂₉F₃N₆O₃ [M+H]⁺: 591.2331, found 591.4819.

### 1.6.7. 1-phenyl-N-(2-(1-(tetrahydro-2H-pyran-2-yl)-1H-indazol-6-yl)-1H-benzo[di]imidazol-5-yl)-5-(trifluoromethyl)-1H-pyrazole-4-carboxamide (7g)

A mixture of 1-phenyl-N-(2-(1-(tetrahydro-2H-pyran-2-yl)-1H-indazol-6-yl)-1H-benzo[di]imidazol-5-yl)-5-(trifluoromethyl)-1H-pyrazole-4-carboxamide was separated in a similar manner to the synthesis of Compound 7a described above to obtain Compound 7g as pure solid in 85.5% yield. ¹H NMR (400 MHz, DMSO-d₆) δ 12.94-12.89 (s, 1H), 10.62-10.53 (s, 1H), 8.58 (s, 1H), 8.43 (d, J=13.8 Hz, 2H), 8.34 (s, 1H), 8.21-8.11 (s, 1H), 7.97-7.91 (m, 1H), 7.87 (d, J=8.8 Hz, 1H), 7.68-7.50 (m, 1H), 7.40 (d, J=8.6 Hz, 1H), 5.79 (m, 1H), 4.06-3.99 (m, 1H), 3.81-3.71 (m, 1H), 2.27-2.17 (m, 1H), 2.14-2.05 (m, 1H), 2.02-1.93 (m, 1H), 1.82-1.71 (m, 1H), 1.66-1.57 (m, 2H). LC/MS (ESI+, m/z) calcd for C₃₀H₂₄F₃N₇O₂[M+H]⁺: 572.2022, found 572.4773.

### 1.6.8. 5-(tert-butyl)-N-(2-(1-(tetrahydro-2H-pyran-2-yl)-1H-indazol-6-yl)-1H-benzo[di]imidazole-5-yl)isoxazole-3-carboxamide (7h)

A mixture of 5-(tert-butyl)-N-(2-(1-(tetrahydro-2H-pyran-2-yl)-1H-indazol-6-yl)-1H-benzo[di]imidazole-5-yl)isoxazole-3-carboxamide was separated in a similar manner to the synthesis of Compound 7a described above to obtain Compound 7h as pure solid in 32.1% yield. ¹H NMR (400 MHz, DMSO-d₆) δ 12.95 (s, 1H), 10.67 (s, 1H), 8.60 (s, 1H), 8.43 (s, 1H), 8.24-8.16 (s, 1H), 7.96-7.87 (m, 2H), 7.65 (d, 1H), 7.53 (dd, J=8.7, 1.9 Hz, 1H), 6.73 (d, J=2.2 Hz, 1H), 5.81 (dd, J=9.6, 2.5 Hz, 2H), 4.04 (d, J=11.3 Hz, 1H), 3.83-3.73 (m, 1H), 2.22 (m, 1H), 2.15-2.08 (m, 1H), 2.05-1.97 (m, 1H), 1.82-1.74 (m, 1H), 1.65 (m, 2H), 1.39 (s, 9H). LC/MS (ESI+, m/z) calcd for C₂₇H₂₈N₆O₃ [M+H]⁺: 485.2301, found 485.1799.

### 1.6.9. 4-chloro-N-(2-(1-(tetrahydro-2H-pyran-2-yl)-1H-indazol-6-yl)-1H-benzo[di]imidazol-5-yl)-3-(trifluoromethyl)benzamide (7i)

A mixture of 4-chloro-N-(2-(1-(tetrahydro-2H-pyran-2-yl)-1H-indazol-6-yl)-1H-benzo[di]imidazol-5-yl)-3-(trifluoromethyl)benzamide was separated in a similar manner to the synthesis of Compound 7a described above to obtain Compound 7i as pure solid in 17.9% yield. ¹H NMR (400 MHz, DMSO-d₆) δ 12.93 (s, 1H), 10.57 (s, 1H), 8.58 (s, 1H), 8.42 (m, =7.4 Hz, 2H), 8.31 (dd, J=8.4, 1.8 Hz, 1H), 8.23-8.14 (s, sH), 7.94 (m, 2H), 7.87 (d, J=8.7 Hz, 1H), 7.66-7.55 (dd, 1H), 7.55-7.47 (d, J=8.8 Hz, 1H), 5.80 (dd, J=9.6, 2.7 Hz, 1H), 4.03 (d, J=11.2 Hz, 1H), 3.80-3.72 (m, 1H), 2.22 (m, 1H), 2.13-2.06 (m, 1H), 2.04-1.90 (m, 2H), 1.76 (m, 1H), 1.63 (m, 1H). LC/MS (ESI+, m/z) calcd for C₂₇H₂₁ClF₃N₅O₂ [M+H]⁺: 540.1414, found 540.4564.

### 1.6.10. 3,4-dichloro-N-(2-(1-(tetrahydro-2H-pyran-2-yl)-1H-indazol-6-yl)-1H-benzo[di]imidazol-5-yl)benzamide (7j)

A mixture of 3,4-dichloro-N-(2-(1-(tetrahydro-2H-pyran-2-yl)-1H-indazol-6-yl)-1H-benzo[di]imidazol-5-yl)benzamide was separated in a similar manner to the synthesis of Compound 7a described above to obtain Compound 7j as pure solid in 30.0% yield. LC/MS (ESI+, m/z) calcd for C₂₆H₂₁Cl₂N₅O₂ [M+H]⁺: 506.1151, found 506.4985.

### 1.6.11. (E)-3-(4-methoxyphenyl)-N-(2-(1-(tetrahydro-2H-pyran-2-yl)-1H-indazol-6-yl)-1H-benzo[di]imidazol-5-yl)acrylamide (7k)

A mixture of (E)-3-(4-methoxyphenyl)-N-(2-(1-(tetrahydro-2H-pyran-2-yl)-1H-indazol-6-yl)-1H-benzo[di]imidazol-5-yl)acrylamide was separated in a similar manner to the synthesis of Compound 7a described above to obtain Compound 7k as pure solid in 78.8% yield. ¹H NMR (400 MHz, DMSO-d₆) δ 12.97 (s, 1H), 10.24-10.14 (s, 1H), 8.49 (s, 1H), 8.35-8.19 (s, 1H), 8.19 (s, 1H), 8.00 (d, J=7.4 Hz, 1H), 7.92 (d, J=8.4 Hz, 1H), 7.64-7.30 (m, 5H), 7.02 (d, J=8.8 Hz, 2H), 6.75 (d, J=15.7 Hz, 2H), 5.98-5.91 (m, 1H), 3.96 (d, J=11.2 Hz, 1H), 3.84 (d, J=7.0 Hz, 1H), 3.81 (s, 3H), 2.13-1.87 (m, 3H), 1.79 (m, 1H), 1.64 (s, 2H). LC/MS (ESI+, m/z) calcd for C₂₉H₂₇N₅O₃ [M+H]⁺: 494.2192, found 494.2188.

### 1.6.12. 1-(5-(tert-butyl)isoxazol-3-yl)-3-(2-(1-(tetrahydro-2H-pyran-2-yl)-1H-indazol-6-yl)-1H-benzo[di]imidazol-5-yl)urea (7l)

5-(tert-butyl)isoxazol-3-amine (10.1 mg, 0.072 mmol) was dissolved in THF (0.36 ml) and added with DIPEA (15.3 μl, 0.072 mmol). The reaction mixture was cooled to 0° C. and added with 4-nitrophenyl carbonochloridate (8.71 mg, 0.0432 mmol). The reaction mixture was stirred at 0° C. for 30 minutes, and then added with Compound 5 (12 mg, 0.036 mmol) and DIPEA (15.3 μl, 0.072 mmol). The reaction mixture was stirred at 50° C. for 16 hours. After completion, the reaction mixture was cooled to ambient temperature, quenched with MeOH and concentrated. The concentrated crude product was purified by flash column chromatography to obtain a desired product as pure solid (4.5 mg). A mixture of 1-(5-(tert-butyl)isoxazol-3-yl)-3-(2-(1-(tetrahydro-2H-pyran-2-yl)-1H-indazol-6-yl)-1H-benzo[di]imidazol-5-yl)urea was separated as pure solid in 25.0% yield. ¹H NMR (400 MHz, DMSO-d₆) δ 12.82 (s, 1H), 9.46 (s, 1H), 8.92-8.79 (s, 1H), 8.57-8.09 (s, 2H), 7.96-7.82 (m, 3H), 7.58-7.45 (d, J=8.5 Hz, 1H), 7.23-7.04 (dd, J=8.6, 1.9 Hz, 1H), 6.53 (s, 1H), 5.79 (m, 1H), 4.56 (t, J=5.5 Hz, 1H), 4.44 (t, J=5.5 Hz, 1H), 4.02 (d, J=11.2 Hz, 1H), 3.79-3.72 (m, 1H), 2.26-2.15 (m, 1H), 2.13-2.06 (m, 1H), 1.97 (m, 2H), 1.31 (s, 9H). LC/MS (ESI+, m/z) calcd for C₂₇H₂₉N₇O₃ [M+H]⁺: 500.2410, found 500.4486.

### 1.6.13. 1-(3,4-dichlorophenyl)-3-(2-(1-(tetrahydro-2H-pyran-2-yl)-1H-indazol-6-yl)-1H-benzo[di]imidazole-5-yl)urea (7m)

A mixture of 1-(3,4-dichlorophenyl)-3-(2-(1-(tetrahydro-2H-pyran-2-yl)-1H-indazol-6-yl)-1H-benzo[di]imidazole-5-yl)urea was separated in a similar manner to the synthesis of Compound 7l described above to obtain Compound 7m as pure solid in 41.6% yield. ¹H NMR (400 MHz, DMSO-d₆) δ 12.81 (s, 1H), 8.97 (s, 1H), 8.88-8.74 (s, 1H), 8.57 (s, 1H), 8.40 (s, 1H), 7.92-7.81 (m, 4H), 7.58-7.45 (d, J=8.6 Hz, 1H), 7.53 (d, J=8.8 Hz, 1H), 7.36 (dd, J=8.8, 2.5 Hz, 1H), 7.27-7.06 (dd, J=8.6, 1.6 Hz, 1H), 5.79 (dd, J=9.6, 2.6 Hz, 1H), 4.02 (d, J=12.1 Hz, 1H), 3.80-3.71 (m, 1H), 2.21 (m, 1H), 2.14-2.06 (m, 1H), 2.03-1.95 (m, 1H), 1.81-1.72 (m, 1H), 1.63 (m, 2H). LC/MS (ESI+, m/z) calcd for C₂₆H₂₂Cl₂N₆O₂ [M+H]⁺: 521.1260, found 521.0828.

### 1.6.14. 3-(3-(dimethylamino)pyrrolidin-1-yl)-N-(2-(1-(tetrahydro-2H-pyran-2-yl)-1H-indazol-6-yl)-1H-benzo[di]imidazol-5-yl)-5-(trifluoromethyl)benzamide (7n)

A mixture of 3-(3-(dimethylamino)pyrrolidin-1-yl)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7n as pure solid in 52.3% yield. m.p. 138-168° C. ¹H NMR (400 MHz, DMSO-d₆) δ 13.04 (s, 1H), 10.39 (s, 1H), 8.52 (s, 1H), 8.28 (s, 1H), 8.24-8.18 (s, 1H), 8.07-8.01 (m, 1H), 7.96 (d, J=8.4 Hz, 1H), 7.69-7.58 (m, 1H), 7.58-7.48 (m, 2H), 7.36 (s, 1H), 6.96 (s, 1H), 6.02-5.94 (m, 1H), 3.98 (d, J=11.4 Hz, 1H), 3.83 (m, 1H), 3.62-3.54 (m, 2H), 3.21-3.16 (m, 1H), 2.89-2.82 (m, 1H), 2.25 (s, 6H), 2.22 (m, 2H), 2.12-2.04 (m, 2H), 1.97-1.77 (m, 3H), 1.66 (s, 2H). LC/MS (ESI+, m/z) calcd for C₃₃H₃₄F₃N₇O₂ [M+H]⁺: 618.2804, found 618.3084.

### 1.6.15. 3-(3-(dimethylamino)pyrrolidin-1-yl)-N-(2-(1-(tetrahydro-2H-pyran-2-yl)-1H-indazol-6-yl)-1H-benzo[di]imidazol-5-yl)-5-(trifluoromethyl)benzamide (7o)

A mixture of 3-(3-(dimethylamino)pyrrolidin-1-yl)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7n as pure solid in 45.9% yield. ¹H NMR (400 MHz, DMSO-d₆) δ 12.91 (s, 1H), 10.34 (s, 1H), 8.58 (s, 1H), 8.43 (s, 1H), 8.21-8.13 (d, J=1.8 Hz, 1H), 7.97-7.91 (m, 1H), 7.87 (d, J=8.8 Hz, 1H), 7.65-7.52 (d, J=8.7 Hz, 1H), 7.59-7.47 (m, 2H), 7.34 (s, 1H), 6.93 (s, 1H), 5.80 (dd, J=9.6, 2.6 Hz, 1H), 4.03 (d, J=12.1 Hz, 1H), 3.80-3.72 (m, 1H), 3.65-3.58 (m, 1H), 2.72-2.55 (m, 4H), 2.26-2.17 (m, 2H), 2.14-2.06 (m, 1H), 1.93 (m, 3H), 1.76 (m, 2H), 1.67-1.58 (m, 2H), 1.45 (m, 1H), 1.36-1.27 (m, 1H), 1.00 (t, J=6.8 Hz, 6H). LC/MS (ESI+, m/z) calcd for C₃₅H₃₈F₃N₇O₂[M+H]⁺: 646.3117, found 646.0709.

### 1.6.16. 3-(4-cyclopropylpiperazin-1-yl)-N-(2-(1-(tetrahydro-2H-pyran-2-yl)-1H-indazol-6-yl)-1H-benzo[di]imidazol-5-yl)-5-(trifluoromethyl)benzamide (7p)

A mixture of 3-(4-cyclopropylpiperazin-1-yl)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7p as pure solid in 60.2% yield. ¹H NMR (400 MHz, DMSO-d₆) δ 13.04 (s, 1H), 10.46-10.40 (s, 1H), 8.52 (s, 1H), 8.28-8.18 (s, 2H), 8.03 (d, J=7.9 Hz, 1H), 7.95 (d, J=8.4 Hz, 1H), 7.77 (s, 1H), 7.70-7.50 (m, 3H), 7.39 (s, 1H), 5.97 (d, J=9.6 Hz, 1H), 3.97 (d, J=11.4 Hz, 1H), 3.91-3.80 (m, 1H), 3.35-3.27 (m, 4H), 2.78-2.65 (m, 4H), 2.07 (m, 2H), 1.90-1.77 (m, 1H), 1.74-1.61 (m, 3H), 1.20 (ddd, J=17.7, 15.6, 11.0 Hz, 1H), 0.51-0.44 (m, 2H), 0.42-0.33 (m, 2H). LC/MS (ESI+, m/z) calcd for C₃₄H₃₄F₃N₇O₂ [M+H]⁺: 630.2804, found 630.3760.

### 1.6.17. 3-((4-cyclopropylpiperazin-1-yl)methyl)-N-(2-(1-(tetrahydro-2H-pyran-2-yl)-1H-indazol-6-yl)-1H-benzo[di]imidazol-5-yl)-5-(trifluoromethyl)benzamide (7q)

A mixture of 3-((4-cyclopropylpiperazin-1-yl)methyl)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7q as pure solid in 20.0% yield. ¹H NMR (400 MHz, DMSO-d₆) δ 13.06-13.04 (s, 1H), 10.57-10.51 (s, 1H), 8.54-8.51 (s, 1H), 8.31-8.20 (m, 4H), 8.03 (dd, J=11.5, 4.3 Hz, 1H), 7.96 (d, J=8.4 Hz, 1H), 7.88 (s, 1H), 7.70-7.59 (d, J=8.7 Hz, 1H), 7.59-7.51 (dd, J=8.7, 1.8 Hz, 1H), 6.02-5.94 (m, TOH), 3.98 (d, J=11.3 Hz, 1H), 3.89-3.81 (m, 1H), 3.67 (s, 2H), 2.58 (m, 4H), 2.41 (m, 5H), 2.13-2.03 (m, 2H), 1.82 (m, 1H), 1.74-1.52 (m, 4H), 0.46-0.37 (m, 2H), 0.29 (d, 2H). LC/MS (ESI+, m/z) calcd for C₃₅H₃₆F₃N₇O₂[M+H]⁺: 644.2961, found 644.4907.

### 1.6.18. 3-((4-ethylpiperazin-1-yl)methyl)-N-(2-(1-(tetrahydro-2H-pyran-2-yl)-1H-indazol-6-yl)-1H-benzo[di]imidazol-5-yl)-5-(trifluoromethyl)benzamide (7r)

A mixture of 3-((4-ethylpiperazin-1-yl)methyl)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7r as pure solid in 29.5% yield. ¹H NMR (400 MHz, DMSO-d₆) δ 13.02 (s, 1H), 10.50 (s, 1H), 8.48 (s, 1H), 8.28-8.14 (m, 4H), 8.02-7.96 (m, 1H), 7.91 (d, J=8.4 Hz, 1H), 7.84 (s, 1H), 7.66 (d, J=8.7 Hz, 1H), 7.54-7.45 (m, 2H), 5.99-5.90 (m, 1H), 3.93 (d, J=11.4 Hz, 1H), 3.80 (dd, J=15.3, 9.4 Hz, 1H), 3.64 (s, 2H), 2.45 (m, TOH), 2.36-2.24 (m, 4H), 2.10-2.00 (m, 2H), 1.84-1.74 (m, 1H), 1.62 (s, 2H). LC/MS (ESI+, m/z) calcd for C₃₄H₃₆F₃N₇O₂[M+H]⁺: 632.2961, found 632.8558.

### 1.6.19. 3-((1-methylpiperidin-4-yl)amino)-N-(2-(1-(tetrahydro-2H-pyran-2-yl)-1H-indazol-6-yl)-1H-benzo[di]imidazol-5-yl)-5-(trifluoromethyl)benzamide (7s)

A mixture of 3-((1-methylpiperidin-4-yl)amino)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7s as pure solid in 41.0% yield. ¹H NMR (400 MHz, DMSO-d₆) δ 12.97 (s, 1H), 10.36 (s, 1H), 8.59 (d, J=0.6 Hz, 1H), 8.45 (s, 1H), 8.20 (s, 1H), 7.96 (dd, J=8.8, 1.2 Hz, 1H), 7.88 (dd, J=8.8, 0.6 Hz, 1H), 7.61 (s, 1H), 7.52 (d, J=7.8 Hz, 1H), 7.39 (d, J=5.4 Hz, 2H), 7.05 (s, 1H), 6.31 (d, J=8.0 Hz, 1H), 5.82 (dd, J=9.6, 2.6 Hz, 1H), 4.10-4.01 (m, 1H), 3.83-3.73 (m, 1H), 3.37 (s, 2H), 2.75 (d, J=11.6 Hz, 2H), 2.27 (dd, J=12.6, 3.4 Hz, 1H), 2.20 (s, 3H), 2.14-1.98 (m, 4H), 1.93 (d, J=10.8 Hz, 2H), 1.83-1.75 (m, 1H), 1.65 (dt, J=11.7, 4.1 Hz, 2H), 1.46 (dd, J=20.3, 10.1 Hz, 2H). LC/MS (ESI+, m/z) calcd for C₃₃H₃₄F₃N₇O₂[M+H]⁺: 618.2804, found 618.5965.

### 1.6.20. 3-((1-ethylpiperidin-4-yl)amino)-N-(2-(1-(tetrahydro-2H-pyran-2-yl)-1H-indazol-6-yl)-1H-benzo[di]imidazol-5-yl)-5-(trifluoromethyl)benzamide (7t)

A mixture of 3-((1-ethylpiperidin-4-yl)amino)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7t as pure solid in 25.8% yield. ¹H NMR (400 MHz, DMSO-d₆) δ 13.22 (s, 1H), 10.33 (s, 1H), 8.51 (s, 1H), 8.18 (s, 2H), 8.03 (dd, J=8.5, 1.1 Hz, 1H), 7.91 (d, J=8.4 Hz, 1H), 7.60 (d, J=8.6 Hz, 1H), 7.53-7.47 (m, 1H), 7.37 (s, 2H), 7.02 (s, 1H), 6.29 (d, J=8.0 Hz, 1H), 5.92 (dd, J=9.6, 2.0 Hz, 1H), 3.94 (d, J=11.6 Hz, 1H), 3.84-3.76 (m, 1H), 3.51-3.37 (m, 2H), 2.82 (d, J=11.3 Hz, 2H), 2.33 (q, J=7.2 Hz, 2H), 2.04 (dd, J=20.7, 11.0 Hz, 4H), 1.94 (t, J=12.7 Hz, 3H), 1.76 (dd, J=23.7, 14.3 Hz, 2H), 1.63 (s, 2H), 1.00 (t, J=7.2 Hz, 3H).

### 1.6.21. 4-((1-methylpiperidin-4-yl)oxy)-N-(2-(1-(tetrahydro-2H-pyran-2-yl)-1H-indazol-6-yl)-1H-benzo[di]imidazol-5-yl)-3-(trifluoromethyl)benzamide (7u)

A mixture of 4-((1-methylpiperidin-4-yl)oxy)-N-(2-(1-(tetrahydro-2H-pyran-2-yl)-1H-indazol-6-yl)-1H-benzo[di]imidazol-5-yl)-3-(trifluoromethyl)benzamide was separated in a similar manner to the synthesis of Compound 7a described above to obtain Compound 7u as pure solid in 41.3% yield. ¹H NMR (400 MHz, DMSO-d₆) δ 13.08 (s, 1H), 10.38 (s, 1H), 8.52 (s, 1H), 8.34-8.19 (m, 4H), 8.04 (dd, J=8.5, 1.2 Hz, 1H), 7.95 (d, J=8.4 Hz, 1H), 7.64 (d, J=8.6 Hz, 1H), 7.54 (d, J=8.6 Hz, 1H), 7.49 (d, J=9.6 Hz, 1H), 5.97 (dd, J=9.6, 2.1 Hz, 1H), 4.80 (s, 1H), 3.97 (d, J=11.3 Hz, 1H), 3.90-3.80 (m, 1H), 2.51-2.45 (m, 2H), 2.33 (dd, J=12.8, 5.2 Hz, 2H), 2.19 (d, J=7.7 Hz, 3H), 2.07 (dd, J=24.7, 11.1 Hz, 2H), 1.98 (dd, J=16.4, 3.6 Hz, 3H), 1.83-1.72 (m, 3H), 1.66 (s, 2H).

### 1.6.22. 3-((1-methylpiperidin-3-yl)amino)-N-(2-(1-(tetrahydro-2H-pyran-2-yl)-1H-indazol-6-yl)-1H-benzo[di]imidazol-5-yl)-5-(trifluoromethyl)benzamide (7v)

A mixture of 3-((1-methylpiperidin-3-yl)amino)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7v as pure solid in 43.8% yield. ¹H NMR (400 MHz, DMSO-d₆) δ 13.01 (s, 1H), 10.34 (s, 1H), 8.50 (s, 1H), 8.21 (m, 2H), 8.01 (dd, J=11.3, 4.0 Hz, 1H), 7.93 (d, J=8.4 Hz, 1H), 7.66 (d, J=8.7 Hz, 1H), 7.56 (m, 1H), 7.48 (dd, J=8.8, 1.9 Hz, 1H), 7.39 (s, 2H), 7.07 (s, 1H), 6.25 (dd, J=8.3, 4.4 Hz, 1H), 5.99-5.92 (m, 1H), 3.96 (d, J=11.1 Hz, 1H), 3.89-3.78 (m, 1H), 3.58 (dd, J=8.7, 4.2 Hz, 1H), 2.81 (d, J=9.6 Hz, 1H), 2.54 (dd, J=15.0, 6.4 Hz, 1H), 2.12-1.92 (m, 4H), 1.89-1.75 (m, 3H), 1.62 (m, 4H), 1.29-1.17 (m, 2H). LC/MS (ESI+, m/z) calcd for C₃₃H₃₄F₃N₇O₂[M+H]⁺: 618.2804, found 618.3084.

### 1.6.23. 4-((4-ethylpiperazin-1-yl)methyl)-N-(2-(1-(tetrahydro-2H-pyran-2-yl)-1H-indazol-6-yl)-1H-benzo[di]imidazol-5-yl)-3-(trifluoromethyl)benzamide (7w)

A mixture of 4-((4-ethylpiperazin-1-yl)methyl)-N-(2-(1-(tetrahydro-2H-pyran-2-yl)-1H-indazol-6-yl)-1H-benzo[di]imidazol-5-yl)-3-(trifluoromethyl)benzamide was separated in a similar manner to the synthesis of Compound 7a described above to obtain Compound 7w as pure solid in 48.5% yield. ¹H NMR (400 MHz, DMSO-d₆) δ 13.08 (s, 1H), 10.56-10.49 (s, 1H), 8.55-8.52 (s, 1H), 8.29-8.20 (m, 4H), 8.03 (m, 1H), 7.96 (m, 2H), 7.69-7.61 (d, J=1.6 Hz, 1H), 7.58-7.52 (dd, J=8.7, 1.9 Hz, 1H), 6.01-5.95 (m, 1H), 3.98-3.80 (m, 2H), 3.72 (s, 2H), 2.47 (m, 8H), 2.37 (m, 3H), 2.08 (m, 2H), 1.89-1.66 (m, 3H), 1.02 (t, J=7.2 Hz, 3H). LC/MS (ESI+, m/z) calcd for C₃₄H₃₆F₃N₇O₂[M+H]⁺: 632.2961, found 632.6398.

### 1.6.24. 3-((1-methylpiperidin-4-yl)oxy)-N-(2-(1-(tetrahydro-2H-pyran-2-yl)-1H-indazol-6-yl)-1H-benzo[di]imidazol-5-yl)-5-(trifluoromethyl)benzamide (7x)

A mixture of 3-((1-methylpiperidin-4-yl)oxy)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7x as pure solid in 20.7% yield. LC/MS (ESI+, m/z) calcd for C₃₃H₃₃F₃N₆O₃ [M+H]⁺: 619.2644, found 619.4607.

### 1.6.25. 3-((4-ethyl-3,3-dimethylpiperazin-1-yl)methyl)-N-(2-(1-(tetrahydro-2H-pyran-2-yl)-1H-indazol-6-yl)-1H-benzo[di]imidazol-5-yl)-5-(trifluoromethyl)benzamide (7y)

A mixture of 3-((4-ethyl-3,3-dimethylpiperazin-1-yl)methyl)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7y as pure solid in 45.1% yield. ¹H NMR (400 MHz, DMSO-d₆) δ 13.17 (d, J=3.8 Hz, 1H), 10.57 (d, J=28.0 Hz, 1H), 8.57 (d, J=8.0 Hz, 1H), 8.32-8.19 (m, 4H), 8.08-8.03 (m, 1H), 7.98-7.89 (m, 2H), 7.77-7.52 (m, 3H), 5.98 (d, J=9.7 Hz, 1H), 3.97 (d, J=11.3 Hz, 1H), 3.86 (dd, J=9.3, 4.3 Hz, 1H), 3.65 (s, 2H), 2.35 (m, 2H), 2.20 (m, 1H), 2.07 (m, 3H), 1.82 (m, 1H), 1.67 (m, 2H), 1.15-0.92 (m, 12H), 0.88-0.81 (m, 2H). LC/MS (ESI+, m/z) calcd for C₃₆H₄₀F₃N₇O₂[M+H]⁺: 660.3274, found 660.5496.

### 1.6.26. 3-(4-ethyl-3,3-dimethylpiperazin-1-yl)-N-(2-(1-(tetrahydro-2H-pyran-2-yl)-1H-indazol-6-yl)-1H-benzo[di]imidazol-5-yl)-5-(trifluoromethyl)benzamide (7z)

A mixture of 3-(4-ethyl-3,3-dimethylpiperazin-1-yl)-N-(2-(1-(tetrahydro-2H-pyran-2-yl)-1H-indazol-6-yl)-1H-benzo[di]imidazol-5-yl)-5-(trifluoromethyl)benzamide was separated in a similar manner to the synthesis of Compound 7a described above to obtain Compound 7z as pure solid in 71.3% yield. ¹H NMR (400 MHz, DMSO-d₆) δ 13.08 (d, J=6.7 Hz, 1H), 10.45 (d, J=26.0 Hz, 1H), 8.54 (d, J=11.4 Hz, 1H), 8.29 (s, 1H), 8.21 (d, J=5.0 Hz, 1H), 8.04 (d, J=7.5 Hz, 1H), 7.95 (d, J=8.4 Hz, 1H), 7.74 (s, 1H), 7.72-7.56 (m, 2H), 7.52 (d, J=8.6 Hz, 1H), 7.39 (s, 1H), 5.97 (d, J=9.6 Hz, 1H), 3.97 (d, J=11.1 Hz, 1H), 3.84 (dd, J=15.3, 9.1 Hz, 1H), 3.11 (s, 2H), 2.67 (s, 2H), 2.50 (d, J=19.2 Hz, 3H), 2.41 (s, 2H), 2.08 (t, J=13.1 Hz, 2H), 1.81 (d, J=8.8 Hz, 1H), 1.65 (s, 2H), 1.09 (s, 6H), 1.03 (t, J=6.8 Hz, 3H). LC/MS (ESI+, m/z) calcd for C₃₅H₃₈F₃N₇O₂ [M+H]⁺: 646.3117, found 646.5791.

### 1.7. Synthesis of Benzimidazole Derivatives (8a-8z) of the Present Disclosure

### 1.7.1. N-(2-(1H-indazol-6-yl)-1H-benzo[di]imidazol-5-yl)-3-(4-methylpiperazin-1-yl)-5-(trifluoromethyl)benzamide (8a)

A solution of Compound 7a (4.6 mg 0.00762 mmol) was added to 5% HCl in EtOH (0.0762 ml to 0.174 ml). The reaction mixture was stirred at room temperature until Compound 7a disappeared on TLC. After completion of the reaction, the solvent was removed in vacuo. The reaction mixture was diluted with ethyl acetate and washed with a 1 M NaOH aqueous solution. The organic layer was dried over Na₂SO₄. The concentrated crude product was purified by flash column chromatography to obtain a desired product as pure solid (2.6 mg). A mixture of N-(2-(1H-indazol-6-yl)-1H-benzo[di]imidazol-5-yl)-3-(4-methylpiperazin-1-yl)-5-(trifluoromethyl)benzamide was separated as pure solid in 50.0% yield. m.p. 228-230° C. ¹H NMR (400 MHz, DMSO-d₆) δ 13.42 (s, 1H), 13.05 (s, 1H), 10.42 (s, 1H), 8.36 (s, 1H), 8.26-8.13 (m, 2H), 7.98 (s, 1H), 7.93 (s, 1H), 7.78 (s, 1H), 7.67 (s, 1H), 7.65-7.47 (m, 2H), 7.40 (s, 1H), 3.36 (m, 4H), 2.54 (m, 4H), 2.27 (s, 3H). ¹³C NMR (101 MHz, DMSO-d₆) δ 164.9, 152.3, 151.8, 144.3, 141.2, 141.2, 140.5, 137.5, 135.5, 134.7, 134.7, 134.2, 132.6, 126.0, 121.6, 121.6, 119.6, 119.0, 119.0, 117.8, 114.0, 108.3, 54.9, 47.9, 46.2. HRMS (ESI+) calcd for C₂₇H₂₄F₃N₇O [M+H]⁺: 520.2073, found 520.1105.

### 1.7.2. N-(2-(1H-indazol-6-yl)-1H-benzo[di]imidazol-5-yl)-3-(4-methyl-1H-imidazol-1-yl)-5-(trifluoromethyl)benzamide (8b)

A mixture of N-(2-(1H-indazol-6-yl)-1H-benzo[di]imidazol-5-yl)-3-(4-methyl-1H-imidazol-1-yl)-5-(trifluoromethyl)benzamide was separated in a similar manner to the synthesis of Compound 8a described above to obtain Compound 8b as pure solid in 8.6% yield. m.p. 239-241° C. ¹H NMR (400 MHz, DMSO-d₆) δ 13.42 (s, 1H), 13.04 (s, 1H), 10.59 (d, J=23.7 Hz, 1H), 8.52 (s, 1H), 8.45 (d, J=1.4 Hz, 1H), 8.36 (d, J=12.2 Hz, 1H), 8.25 (d, J=17.4 Hz, 2H), 8.19 (d, J=8.2 Hz, 1H), 7.98 (s, 1H), 7.94 (d, J=8.4 Hz, 1H), 7.76 (s, 1H), 7.70 (d, J=8.7 Hz, 1H), 7.59 (s, 1H), 7.51 (d, J=7.4 Hz, 1H), 2.23 (d, J=0.8 Hz, 3H). 13C NMR (101 MHz, DMSO-d₆) δ 161.4, 152.9, 145.7, 142.3, 140.5, 139.4, 138.4, 138.2, 137.6, 136.8, 136.3, 136.0, 133.3, 131.6, 128.7, 127.4, 124.0, 121.6, 121.6, 121.6, 119.5, 118.8, 113.9, 111.7, 109.2, 14.5. HRMS (ESI+) calcd for C₂₆H₁₈F₃N₇O [M+H]⁺: 502.1603, found 502.0331.

### 1.7.3. N-(2-(1H-indazol-6-yl)-1H-benzo[di]imidazol-5-yl)-3-(2-methyl-1H-imidazol-1-yl)-5-(trifluoromethyl)benzamide (8c)

20% TFA (0.0348 ml) was added to a solution of Compound 7c (10.2 mg 0.01742 mmol) and CH₂Cl₂ (0.1742 ml). The reaction mixture was stirred at room temperature until Compound 7c disappeared on TLC. After completion of the reaction, the solvent was removed in vacuo. The reaction mixture was diluted with ethyl acetate and washed with saturated aqueous sodium bicarbonate. The organic layer was dried over Na₂SO₄.

The concentrated crude product was purified by flash column chromatography to obtain a desired product as pure solid (2.1 mg). A mixture of N-(2-(1H-indazol-6-yl)-1H-benzo[di]imidazol-5-yl)-3-(2-methyl-1H-imidazol-1-yl)-5-(trifluoromethyl)benzamide was separated as pure solid in 10.0% yield. m.p. 251-253° C. ¹H NMR (400 MHz, DMSO-d₆) δ 13.41 (s, 1H), 13.05 (d, J=13.6 Hz, 1H), 10.62 (s, 1H), 8.40 (d, J=9.3 Hz, 2H), 8.34 (s, 1H), 8.27 (d, J=1.7 Hz, 1H), 8.17 (d, J=3.7 Hz, 1H), 7.99-7.96 (m, 1H), 7.94 (d, J=8.4 Hz, 1H), 7.69 (d, J=8.8 Hz, 1H), 7.62-7.57 (m, 1H), 7.55 (d, J=1.4 Hz, 1H), 7.50 (dd, J=8.6, 1.9 Hz, 1H), 7.02 (d, J=1.4 Hz, 1H), 2.40 (s, 3H). 13C NMR (101 MHz, DMSO-d₆) δ 165.4, 152.5, 147.4, 144.7, 141.0, 139.5, 138.6, 137.7, 135.3, 134.9, 134.6, 134.2, 132.3, 131.5, 130.7, 129.6, 121.8, 121.3, 120.2, 120.1, 119.3, 119.3, 114.7, 113.1, 108.4, 13.7. HRMS (ESI+) calcd for C₂₆H₂₈F₃N₇O [M+H]⁺: 502.1603, found 502.3932.

### 1.7.4. N-(2-(1H-indazol-6-yl)-1H-benzo[di]imidazol-5-yl)-3-morpholino-5-(trifluoromethyl)benzamide (8d)

A mixture of N-(2-(1H-indazol-6-yl)-1H-benzo[di]imidazol-5-yl)-3-morpholino-5-(trifluoromethyl)benzamide was separated in a similar manner to the synthesis of Compound 8a described above to obtain Compound 8d as pure solid in 99.0% yield. m.p. 195-196° C. ¹H NMR (400 MHz, DMSO-d₆) δ 13.41 (s, 1H), 13.02 (s, 1H), 10.45-10.39 (s, 1H), 8.36 (s, 1H), 8.24-8.16 (s, 1H), 8.17 (s, 1H), 8.01-7.93 (m, 2H), 7.78 (s, 1H), 7.71-7.63 (m, 2H), 7.57-7.47 (s, 1H), 7.42 (s, 1H), 3.83-3.78 (m, 4H), 3.33 (s, 4H). ¹³C NMR (101 MHz, DMSO-d₆) δ 164.8, 152.3, 152.0, 144.3, 141.2, 140.5, 137.5, 135.5, 134.6, 134.2, 128.4, 124.0, 121.6, 119.5, 119.0, 117.7, 116.5, 114.5, 114.4, 113.8, 111.7, 111.4, 108.3, 103.7, 66.4, 48.2. HRMS (ESI+) calcd for C₂₆H₂₁F₃N₆O₂[M+H]⁺: 507.1756, found 507.4708.

### 1.7.5. N-(2-(1H-indazol-6-yl)-1H-benzo[di]imidazol-5-yl)-4-(4-methylpiperazin-1-yl)-3-(trifluoromethyl)benzamide (8e)

A mixture of N-(2-(1H-indazol-6-yl)-1H-benzo[di]imidazol-5-yl)-4-(4-methylpiperazin-1-yl)-3-(trifluoromethyl)benzamide was separated in a similar manner to the synthesis of Compound 8c described above to obtain Compound 8e as pure solid in 76.1% yield. m.p. 160-161° C. ¹H NMR (400 MHz, DMSO-d₆) δ 13.42 (s, 1H), 13.05-13.01 (s, 1H), 10.46-10.40 (s, 1H), 8.36 (d, J=12.3 Hz, 1H), 8.31-8.23 (m, 3H), 8.17 (s, 1H), 7.98 (dd, J=7.4 Hz, 1H), 7.93 (d, J=8.5 Hz, 1H), 7.69-7.63 (m, 2H), 7.58-7.50 (d, J=8.6 Hz, 1H), 3.01 (t, J=4.6 Hz, 4H), 2.54 (t, 3H), 2.30 (s, 3H). ¹³C NMR (101 MHz, DMSO-d₆) δ 164.2, 155.1, 141.1, 140.5, 135.6, 134.2, 133.3, 131.3, 128.4, 127.5, 125.8, 124.8, 124.5, 124.1, 123.1, 121.6, 119.5, 119.0, 116.3, 111.4, 108.3, 103.5, 55.2, 53.0, 46.0. HRMS (ESI+) calcd for C₂₇H₂₄F₃N₇O [M+H]⁺: 520.2073, found 520.4830.

### 1.7.6. N-(2-(1H-indazol-6-yl)-1H-benzo[di]imidazol-5-yl)-4-morpholino-3-(trifluoromethyl)benzamide (8f)

A mixture of N-(2-(1H-indazol-6-yl)-1H-benzo[di]imidazol-5-yl)-4-morpholino-3-(trifluoromethyl)benzamide was separated in a similar manner to the synthesis of Compound 8c described above to obtain Compound 8f as pure solid in 44.4% yield. m.p. 202-204° C. ¹H NMR (400 MHz, DMSO-d₆) δ 13.39 (s, 1H), 12.98 (s, 1H), 10.44 (s, 1H), 8.32 (s, 1H), 8.28 (s, 1H), 8.23 (s, 1H), 8.15 (s, 1H), 7.96 (dd, J=8.6 Hz, 1H), 7.91 (d, J=8.4 Hz, 1H), 7.72-7.61 (m, 2H), 7.51 (m, 2H), 3.80-3.70 (t, 4H), 3.01-2.93 (t, 4H). ¹³C NMR (101 MHz, DMSO-d₆) δ 164.2, 154.8, 152.5, 149.9, 147.6, 140.5, 134.2, 133.4, 131.6, 128.3, 127.4, 127.4, 125.8, 125.1, 124.8, 124.3, 124.0, 123.1, 121.6, 119.5, 116.8, 108.4, 66.9, 53.6. HRMS (ESI+) calcd for C₂₆H₂₁F₃N₆O₂[M+H]⁺: 507.1756, found 507.3987.

### 1.7.7. N-(2-(1H-indazol-6-yl)-1H-benzo[di]imidazol-5-yl)-1-phenyl-5-(trifluoromethyl)-1H-pyrazole-4-carboxamide (8g)

A mixture of N-(2-(1H-indazol-6-yl)-1H-benzo[di]imidazol-5-yl)-1-phenyl-5-(trifluoromethyl)-1H-pyrazole-4-carboxamide was separated in a similar manner to the synthesis of Compound 8c described above to obtain Compound 8g as pure solid in 22.0% yield. m.p. 258-260° C. ¹H NMR (400 MHz, DMSO-d₆) δ 13.39 (s, 1H), 13.01-12.96 (s, 1H), 10.61-10.52 (s, 1H), 8.35-8.31 (m, 2H), 8.21-8.10 (m, 2H), 7.95-7.91 (m, 2H), 7.66-7.39 (m, 7H). ¹³C NMR (101 MHz, DMSO-d₆) δ 159.3, 152.3, 144.3, 141.1, 140.5, 140.3, 135.6, 134.6, 134.2, 133.8, 130.6, 129.9, 128.4, 126.5, 122.3, 121.6, 121.2, 119.5, 119.2, 116.9, 115.6, 108.3, 102.8. HRMS (ESI+) calcd for C₂₅H₁₆F₃N₇O [M+H]⁺: 488.1447, found 488.3489.

### 1.7.8. N-(2-(1H-indazol-6-yl)-1H-benzo[di]imidazol-5-yl)-5-(tert-butyl)isoxazole-3-carboxamide (8h)

A mixture of N-(2-(1H-indazol-6-yl)-1H-benzo[di]imidazol-5-yl)-5-(tert-butyl)isoxazole-3-carboxamide was separated in a similar manner to the synthesis of Compound 8a described above to obtain Compound 8h as pure solid in 22.0% yield. m.p. 147-149° C. ¹H NMR (400 MHz, DMSO-d₆) δ 13.40 (s, 1H), 13.01 (s, 1H), 10.70-10.61 (s, 1H), 8.33 (s, 1H), 8.21-8.15 (m, 2H), 7.96-7.91 (m, 2H), 7.63 (m, 1H), 7.52 (m, 1H), 6.71 (s, 1H), 1.36 (s, 9H). ¹³C NMR (101 MHz, DMSO) δ 159.7, 157.9, 153.0, 152.5, 141.4, 140.5, 135.5, 134.2, 128.3, 124.0, 121.6, 119.5, 119.5, 119.2, 116.2, 111.5, 103.5, 99.3, 33.1, 29.0. HRMS (ESI+) calcd for C₂₂H₂₀N₆O₂ [M+H]⁺: 401.1726, found 401.4507.

### 1.7.9. N-(2-(1H-indazol-6-yl)-1H-benzo[di]imidazol-5-yl)-4-chloro-3-(trifluoromethyl)benzamide (8i)

A mixture of N-(2-(1H-indazol-6-yl)-1H-benzo[di]imidazol-5-yl)-4-chloro-3-(trifluoromethyl)benzamide was separated in a similar manner to the synthesis of Compound 8a described above to obtain Compound 8i as pure solid in 100.0% yield. m.p. 215-217° C. ¹H NMR (400 MHz, DMSO-d₆) δ 13.41 (s, 1H), 13.04-13.01 (s, 1H), 10.62-10.56 (s, 1H) 8.45 (s, 1H), 8.37-8.32 (m, 2H), 8.26-8.25 (m, 2H), 8.00-7.92 (m, 3H), 7.69-7.48 (m, 2H). ¹³C NMR (101 MHz, DMSO-d₆) δ 163.5, 152.4, 144.3, 141.3, 140.5, 135.5, 134.9, 134.5, 134.2, 133.9, 132.4, 128.4, 127.6, 124.0, 121.6, 119.5, 119.1, 117.6, 116.3, 111.5, 108.3, 103.6. HRMS (ESI+) calcd for C₂₂H₁₃ClF₃N₅O [M+H]⁺: 456.0839, found 456.2264.

### 1.7.10. N-(2-(1H-indazol-6-yl)-1H-benzo[di]imidazol-5-yl)-3,4-dichlorobenzamide (8j)

A mixture of N-(2-(1H-indazol-6-yl)-1H-benzo[di]imidazol-5-yl)-3,4-dichlorobenzamide was separated in a similar manner to the synthesis of Compound 8a described above to obtain Compound 8j as pure solid in 76.7% yield. m.p. 255-257° C. ¹H NMR (400 MHz, DMSO-d₆) δ 13.39 (s, 1H), 13.01 (s, 1H), 10.47 (s, 1H), 8.33 (d, J=13.5 Hz, 1H), 8.25-8.15 (m, 3H), 7.97-7.91 (m, 3H), 7.84 (dd, J=8.4, 1.6 Hz, 1H), 7.58 (dd, J=8.6, 1.7 Hz, 1H), 7.48 (dd, J=8.7, 2.0 Hz, 1H). ¹³C NMR (101 MHz, DMSO-d₆) δ 163.5, 152.4, 141.2, 140.5, 136.0, 135.5, 134.7, 134.6, 134.2, 131.8, 131.2, 130.1, 128.5, 124.1, 124.0, 121.6, 119.5, 119.1, 117.6, 116.3, 108.3. HRMS (ESI+) calcd for C₂₁H₁₃Cl₂N₅O [M+H]⁺: 422.0575, found 422.3745.

### 1.7.11. (E)-N-(2-(1H-indazol-6-yl)-1H-benzo[di]imidazol-5-yl)-3-(4-methoxyphenyl)acrylamide (8k)

A mixture of (E)-N-(2-(1H-indazol-6-yl)-1H-benzo[di]imidazol-5-yl)-3-(4-methoxyphenyl)acrylamide was separated in a similar manner to the synthesis of Compound 8c described above to obtain Compound 8k as pure solid in 99.0% yield. m.p. 206-208° C. ¹H NMR (400 MHz, DMSO-d₆) δ 13.39 (s, 1H), 12.97 (s, 1H), 10.19 (s, 1H), 8.32 (s, 1H), 8.18 (s, 1H), 7.95 (d, J=7.7 Hz, 1H), 7.90 (d, J=8.4 Hz, 1H), 7.59-7.48 (s, 5H), 7.29 (d, J=7.8 Hz, 1H), 7.02 (d, J=8.7 Hz, 2H), 6.75 (d, J=15.6 Hz, 1H), 3.81 (s, 3H). ¹³C NMR (101 MHz, DMSO-d₆) δ 164.2, 161.0, 152.1, 140.6, 140.5, 140.0, 137.5, 136.8, 136.1, 136.1, 134.3, 129.8, 127.9, 124.1, 124.0, 121.6, 120.5, 119.5, 119.5, 115.0, 108.4, 55.8. HRMS (ESI+) calcd for C₂₄H₁₉N₅O₂ [M+H]⁺: 410.1617, found 410.5261.

### 1.7.12. 1-(2-(1H-indazol-6-yl)-1H-benzo[di]imidazol-5-yl)-3-(5-(tert-butyl)isoxazol-3-yl)urea (8l)

A mixture of 1-(2-(1H-indazol-6-yl)-1H-benzo[di]imidazol-5-yl)-3-(5-(tert-butyl)isoxazol-3-yl)urea was separated in a similar manner to the synthesis of Compound 8c described above to obtain Compound 8l as pure solid in 72.2% yield. m.p. 184-186° C. ¹H NMR (400 MHz, DMSO-d₆) δ 13.44 (s, 1H), 9.50 (s, 1H), 8.96 (s, 1H), 8.33 (s, 1H), 8.17 (s, 1H), 8.09 (s, 1H), 7.93 (s, 2H), 7.57 (d, J=8.3 Hz, 1H), 7.15 (d, J=8.5 Hz, 1H), 6.53 (s, 1H), 1.31 (s, 9H). ¹³C NMR (101 MHz, DMSO) δ 160.0, 152.6, 152.0, 149.0, 147.2, 146.9, 146.8, 140.6, 140.3, 136.6, 134.2, 130.0, 128.9, 123.5, 119.4, 113.5, 109.3, 92.9, 31.2, 28.9. HRMS (ESI+) calcd for C₂₂H₂₁N₇O₂ [M+H]⁺: 416.1835, found 416.4323.

### 1.7.13. 1-(2-(1H-indazol-6-yl)-1H-benzo[di]imidazol-5-yl)-3-(3,4-dichlorophenyl)urea (8m)

A mixture of 1-(2-(1H-indazol-6-yl)-1H-benzo[di]imidazol-5-yl)-3-(3,4-dichlorophenyl)urea was separated in a similar manner to the synthesis of Compound 8c described above to obtain Compound 8m as pure solid in 56.1% yield. m.p. 144-146° C. ¹H NMR (400 MHz, DMSO-d₆) δ 13.36 (s, 1H), 12.88 (s, 1H), 9.08 (s, 1H), 9.00 (s, 1H), 8.31 (s, 1H), 8.14 (s, 1H), 7.97-7.84 (m, 4H), 7.57 (d, J=8.6 Hz, 1H), 7.52 (dd, J=8.8, 2.5 Hz, 1H), 7.36 (m, 1H), 7.07 (dd, J=8.6, 1.9 Hz, 1H). ¹³C NMR (101 MHz, DMSO) δ 160.0, 140.5, 139.5, 138.8, 134.2, 131.7, 131.5, 131.1, 131.0, 129.7, 128.6, 128.6, 123.8, 121.5, 120.1, 119.7, 119.4, 119.3, 118.8, 114.9, 108.1. HRMS (EST⁺) calcd for C₂₁H₁₄Cl₂N₆O [M+H]⁺: 437.0684, found 437.2479.

### 1.7.14. N-(2-(1H-indazol-6-yl)-1H-benzo[di]imidazol-5-yl)-3-(3-(dimethylamino)pyrrolidin-1-yl)-5-(trifluoromethyl)benzamide (8n)

A mixture of N-(2-(1H-indazol-6-yl)-1H-benzo[di]imidazol-5-yl)-3-(3-(dimethylamino)pyrrolidin-1-yl)-5-(trifluoromethyl)benzamide was separated in a similar manner to the synthesis of Compound 8c described above to obtain Compound 8n as pure solid in 11.0% yield. m.p. 283-285° C. ¹H NMR (400 MHz, DMSO-d₆) δ 13.39 (s, 1H), 13.01 (s, 1H), 10.47 (s, 1H), 8.33 (d, J=13.5 Hz, 1H), 8.25-8.15 (m, 3H), 7.97-7.91 (m, 3H), 7.84 (dd, J=8.4, 1.6 Hz, 1H), 7.58 (dd, J=8.6, 1.7 Hz, 1H), 7.48 (dd, J=8.7, 2.0 Hz, 1H). ¹³C NMR (101 MHz, DMSO-d₆) δ 165.2, 152.4, 148.1, 141.1, 140.5, 137.4, 135.6, 134.7, 134.2, 130.7, 128.3, 126.2, 124.0, 123.5, 121.5, 119.6, 119.0, 116.6, 114.4, 110.7, 110.0, 103.8, 65.5, 52.5, 47.4, 44.4, 30.1. HRMS (ESI+) calcd for C₂₈H₂₆F₃N₇O [M+H]⁺: 534.2229, found 534.4427.

### 1.7.15. N-(2-(1H-indazol-6-yl)-1H-benzo[di]imidazol-5-yl)-3-(3-(diethylamino)pyrrolidin-1-yl)-5-(trifluoromethyl)benzamide (80)

A mixture of N-(2-(1H-indazol-6-yl)-1H-benzo[di]imidazol-5-yl)-3-(3-(diethylamino)pyrrolidin-1-yl)-5-(trifluoromethyl)benzamide was separated in a similar manner to the synthesis of Compound 8c described above to obtain Compound 8o as pure solid in 24.6% yield. m.p. 221-223° C. ¹H NMR (400 MHz, DMSO-d₆) δ 13.38 (s, 1H), 12.98 (s, 1H), 10.37-10.31 (s, 1H), 8.34-8.31 (s, 1H), 8.21-8.09 (m, 2H), 7.97 (dd, J=11.4, 4.2 Hz, 1H), 7.91 (d, J=8.1 Hz, 1H), 7.65-7.50 (d, J=8.5 Hz, 1H), 7.56-7.46 (dd, J=8.6, 1.6 Hz, 1H), 7.52-7.44 (m, 1H), 7.34 (s, 1H), 6.94 (s, 1H), 4.77 (s, 1H), 4.56 (m, 2H), 4.43 (t, J=5.4 Hz, 4H), 2.67 (m, 4H), 1.09 (t, J=7.0 Hz, 6H). ¹³C NMR (101 MHz, DMSO) δ 162.7, 158.9, 150.9, 147.5, 143.6, 141.2, 140.0, 140.0, 137.4, 136.3, 135.7, 135.6, 134.3, 130.0, 129.1, 126.2, 126.1, 119.1, 119.0, 114.4, 110.7, 60.3, 47.1, 43.8, 31.2, 14.4, 11.8. HRMS (ESI+) calcd for C₃₀H₃₀F₃N₇O [M+H]⁺: 562.2542, found 562.5664

### 1.7.16. N-(2-(1H-indazol-6-yl)-1H-benzo[di]imidazol-5-yl)-3-(4-cyclopropylpiperazin-1-yl)-5-(trifluoromethyl)benzamide (8p)

A mixture of N-(2-(1H-indazol-6-yl)-1H-benzo[di]imidazol-5-yl)-3-(4-cyclopropylpiperazin-1-yl)-5-(trifluoromethyl)benzamide was separated in a similar manner to the synthesis of Compound 8c described above to obtain Compound 8p as pure solid in 93.9% yield. m.p. 154-156° C. ¹H NMR (400 MHz, DMSO-d₆) δ 13.41 (s, 1H), 13.01 (s, 1H), 10.43 (s, 1H), 8.36 (s, 1H), 8.19 (m, 2H), 7.99 (dd, J=6.8, 1H), 7.93 (d, J=8.5 Hz, 1H), 7.77 (s, 1H), 7.71-7.57 (m, 2H), 7.52 (d, J=7.1 Hz, 1H), 7.39 (s, 1H), 3.33 (m, 5H), 2.76 (s, 4H), 0.46 (m, 4H). ¹³C NMR (101 MHz, DMSO-d₆) δ 164.8, 151.9, 151.7, 140.5, 137.5, 134.2, 130.8, 130.4, 128.3, 126.0, 124.0, 124.0, 123.3, 121.6, 119.5, 117.9, 117.9, 117.9, 114.1, 114.1, 114.0, 108.4, 52.9, 47.9, 40.0, 38.5. HRMS (ESI+) caled for C₂₉H₂₆F₃N₇O [M+H]⁺: 546.2229, found 546.5882.

### 1.7.17. N-(2-(1H-indazol-6-yl)-1H-benzo[di]imidazol-5-yl)-3-((4-cyclopropylpiperazin-1-yl)methyl)-5-(trifluoromethyl)benzamide (8q)

A mixture of N-(2-(1H-indazol-6-yl)-1H-benzo[di]imidazol-5-yl)-3-((4-cyclopropylpiperazin-1-yl)methyl)-5-(trifluoromethyl)benzamide was separated in a similar manner to the synthesis of Compound 8c described above to obtain Compound 8q as pure solid in 89.7% yield. m.p. 150-152° C. ¹H NMR (400 MHz, DMSO-d₆) δ 13.41 (s, 1H), 13.04-13.01 (s, 1H), 10.56-10.50 (s, 1H), 8.37-8.34 (s, 1H), 8.26-8.17 (m, 4H), 7.98-7.93 (m, 2H), 7.88 (s, 1H), 7.68-7.50 (m, 2H), 3.67 (s, 2H), 2.58 (m, 4H), 2.41 (m, 5H), 0.41-0.29 (m, 4H). ¹³C NMR (101 MHz, DMSO-d₆) δ 164.4, 158.4, 140.5, 136.7, 134.2, 132.6, 132.6, 132.6, 130.0, 129.8, 129.5, 129.5, 128.6, 128.3, 125.9, 125.4, 124.0, 123.2, 121.6, 119.5, 116.4, 108.4, 61.3, 53.1, 52.7, 38.4, 6.1. HRMS (ESI+) calcd for C₃₀H₂₈F₃N₇O [M+H]⁺: 560.2386, found 560.7390.

### 1.7.18. N-(2-(1H-indazol-6-yl)-1H-benzo[di]imidazol-5-yl)-3-((4-ethylpiperazin-1-yl)methyl)-5-(trifluoromethyl)benzamide (8r)

A mixture of N-(2-(1H-indazol-6-yl)-1H-benzo[di]imidazol-5-yl)-3-((4-ethylpiperazin-1-yl)methyl)-5-(trifluoromethyl)benzamide was separated in a similar manner to the synthesis of Compound 8a described above to obtain Compound 8r as pure solid in 65.5% yield. m.p. 265-267° C. ¹H NMR (400 MHz, DMSO-d₆) δ 10.46 (s, 1H), 8.35 (s, 1H), 8.27 (m, 2H), 8.15 (s, 1H), 8.13 (s, 1H), 7.99 (dd, J=8.5, 1.3 Hz, 1H), 7.94 (d, J=7.8 Hz, 1H), 7.87 (d, J=8.5 Hz, 1H), 7.57 (d, J=8.6 Hz, 1H), 7.46 (dd, J=8.6, 1.6 Hz, 1H), 3.69 (s, 2H), 3.34 (s, 4H), 2.44 (s, 4H), 2.35-2.30 (m, 2H), 0.98 (m, 3H). ¹³C NMR (101 MHz, DMSO-d₆) δ 164.4, 152.4, 141.1, 141.1, 136.6, 135.6, 134.6, 134.2, 132.5, 129.8, 129.5, 128.3, 125.9, 124.0, 123.4, 123.4, 123.4, 121.6, 119.5, 119.1, 116.4, 103.7, 61.4, 52.9, 52.7, 52.0, 12.3. HRMS (ESI+) calcd for C₂₉H₂₈F₃N₇O [M+H]⁺: 548.2386, found 548.4866.

### 1.7.19. N-(2-(1H-indazol-6-yl)-1H-benzo[di]imidazol-5-yl)-3-((1-methylpiperidin-4-yl)amino)-5-(trifluoromethyl)benzamide (8s)

A mixture of N-(2-(1H-indazol-6-yl)-1H-benzo[di]imidazol-5-yl)-3-((1-methylpiperidin-4-yl)amino)-5-(trifluoromethyl)benzamide was separated in a similar manner to the synthesis of Compound 8a described above to obtain Compound 8s as pure solid in 96.7% yield. m.p. 175-177° C. ¹H NMR (400 MHz, DMSO-d₆) δ 13.40 (s, 1H), 12.99 (s, 1H), 10.35 (s, 1H), 8.33 (s, 1H), 8.19-8.15 (m, 2H), 7.97-7.89 (m, 2H), 7.62-7.37 (m, 4H), 7.05 (s, 1H), 6.50-6.31 (d, 1H), 3.64 (m, 1H), 3.46-3.38 (m, 2H), 2.85 (d, 2H), 2.26 (s, 3H), 1.99-1.92 (m, 2H), 1.51-1.42 (m, 2H). ¹³C NMR (101 MHz, DMSO-d₆) δ 165.3, 156.1, 154.8, 149.0, 141.1, 140.6, 137.7, 135.5, 134.3, 134.2, 128.4, 127.1, 126.2, 124.0, 121.6, 119.6, 119.5, 115.0, 111.2, 111.1, 110.7, 103.6, 60.2, 54.2, 46.1, 31.5. HRMS (ESI+) calcd for C₂₈H₂₆F₃N₇O [M+H]⁺: 534.2229, found 534.2986.

### 1.7.20. N-(2-(1H-indazol-6-yl)-1H-benzo[di]imidazol-5-yl)-3-((1-ethylpiperidin-4-yl)amino)-5-(trifluoromethyl)benzamide (8t)

A mixture of N-(2-(1H-indazol-6-yl)-1H-benzo[di]imidazol-5-yl)-3-((1-ethylpiperidin-4-yl)amino)-5-(trifluoromethyl)benzamide was separated in a similar manner to the synthesis of Compound 8c described above to obtain Compound 8t as pure solid in 21.2% yield. m.p. 166-168° C. ¹H NMR (400 MHz, DMSO-d₆) δ 13.41 (s, 1H), 13.00 (s, 1H), 10.37-10.30 (s, 1H), 8.35 (s, 1H), 8.22-8.15 (s, 1H), 8.17 (s, 1H), 8.01-7.91 (m, 2H), 7.66-7.53 (d, J=8.7 Hz, 1H), 7.57-7.47 (dd, J=1.5 Hz, 1H), 7.40 (m, 2H), 7.06 (s, 1H), 6.34 (s, 1H), 4.58 (s, 1H), 2.51 (q, 2h) 2.00-1.91 (m, 4H), 1.47 (s, 4H), 1.12 (t, J=7.0 Hz, 3H). ¹³C NMR (101 MHz, DMSO-d₆) δ 163.7, 150.8, 148.8, 139.1, 138.5, 134.8, 134.6, 134.2, 131.5, 129.0, 126.7, 125.7, 124.6, 121.6, 121.0, 120.3, 118.9, 117.6, 116.6, 116.4, 115.8, 113.6, 63.0, 53.1, 51.7, 30.4, 14.3. HRMS (ESI+) calcd for C₂₉H₂₈F₃N₇O [M+H]⁺: 548.2386, found 548.5326.

### 1.7.21. N-(2-(1H-indazol-6-yl)-1H-benzo[di]imidazol-5-yl)-3-((1-methylpiperidin-4-yl)oxy)-5-(trifluoromethyl)benzamide (8u)

A mixture of N-(2-(1H-indazol-6-yl)-1H-benzo[di]imidazol-5-yl)-3-((1-methylpiperidin-4-yl)oxy)-5-(trifluoromethyl)benzamide was separated in a similar manner to the synthesis of Compound 8a described above to obtain Compound 8u as pure solid in 85.9% yield. m.p. 293-295° C. ¹H NMR (400 MHz, DMSO-d₆) δ 13.40 (s, 1H), 13.00 (s, 1H), 10.44 (s, 1H), 8.34 (s, 1H), 8.21 (s, 1H), 8.15 (s, 1H), 7.95 (dd, 1H), 7.91 (d, J=8.4 Hz, 1H), 7.87 (s, 1H), 7.84 (s, 1H), 7.70-7.61 (m, 1H), 7.55 (dd, J=10.4 Hz, 1H), 7.49 (s, 1H), 4.70-4.63 (m, 1H), 2.61 (d, J=6.5 Hz, 2H), 2.28-2.17 (m, 5H), 1.95 (d, J=13.4 Hz, 2H), 1.75-1.66 (m, 2H). ¹³C NMR (101 MHz, DMSO) δ 165.4, 158.1, 154.1, 148.1, 146.8, 144.9, 138.2, 134.6, 134.5, 134.2, 132.6, 132.6, 132.4, 132.0, 131.1, 130.6, 129.9, 125.2, 119.5, 115.4, 112.1, 110.9, 52.7, 46.3, 30.8, 29.5. HRMS (ESI+) calcd for C₂₈H₂₅F₃N₆O₂[M+H]⁺: 535.2069, found 535.3787.

### 1.7.22. N-(2-(1H-indazol-6-yl)-1H-benzo[di]imidazol-5-yl)-3-((1-methylpiperidin-3-yl)amino)-5-(trifluoromethyl)benzamide (8v)

A mixture of N-(2-(1H-indazol-6-yl)-1H-benzo[di]imidazol-5-yl)-3-((1-methylpiperidin-3-yl)amino)-5-(trifluoromethyl)benzamide was separated in a similar manner to the synthesis of Compound 8a described above to obtain Compound 8v as pure solid in 100.0% yield. m.p. 213-214° C. ¹H NMR (400 MHz, DMSO-d₆) δ 13.42 (s, 1H), 13.02 (s, 1H), 10.35 (s, 1H), 8.36 (s, 1H), 8.22 (s, 1H), 8.16 (s, 1H), 8.03-7.96 (m, 1H), 7.93 (d, J=8.5 Hz, 1H), 7.66-7.59 (m, 1H), 7.57-7.48 (m, 1H), 7.41 (s, 2H), 7.09 (s, 1H), 6.29 (d, J=8.3 Hz, 1H), 3.64-3.57 (m, 1H), 2.85 (d, J=9.1 Hz, 1H), 2.59 (d, 1H), 2.22 (s, 3H), 2.02 (m, 1H), 1.91 (m, 2H), 1.74 (m, 1H), 1.66-1.57 (m, 1H), 1.28 (m, 1H). ¹³C NMR (101 MHz, DMSO-d₆) δ 165.3, 152.3, 149.0, 141.1, 140.5, 137.7, 135.5, 134.8, 134.2, 130.3, 128.4, 126.1, 121.6, 119.5, 119.0, 116.4, 114.7, 111.4, 110.8, 110.8, 108.3, 103.6, 60.8, 55.7, 48.7, 46.5, 29.7, 23.7. HRMS (ESI+) calcd for C₂₈H₂₆F₃N₇O [M+H]⁺: 534.2229, found 534.3707.

### 1.7.23. N-(2-(1H-indazol-6-yl)-1H-benzo[di]imidazol-5-yl)-4-((4-ethylpiperazin-1-yl)methyl)-3-(trifluoromethyl)benzamide (8w)

A mixture of N-(2-(1H-indazol-6-yl)-1H-benzo[di]imidazol-5-yl)-4-((4-ethylpiperazin-1-yl)methyl)-3-(trifluoromethyl)benzamide was separated in a similar manner to the synthesis of Compound 8c described above to obtain Compound 8w as pure solid in 66.8% yield. m.p. 164-166° C. ¹H NMR (400 MHz, DMSO-d₆) δ 13.42 (s, 1H), 13.08-13.05 (s, 1H), 10.55-10.48 (s, 1H), 8.36-8.19 (m, 4H), 8.15 (s, 1H), 8.00-7.89 (m, 3H), 7.65-7.50 (m, 2H), 3.73 (s, 2H), 3.32 (m, 4H), 2.49 (m, 6H), 1.23 (s, 3H). ¹³C NMR (101 MHz, DMSO) δ 164.4, 156.8, 152.4, 141.2, 140.5, 134.8, 134.2, 132.1, 132.1, 131.2, 128.4, 127.9, 127.6, 125.7, 124.0, 123.3, 121.6, 119.5, 119.1, 116.4, 111.5, 103.6, 57.6, 52.0, 51.7, 29.5, 11.2. HRMS (ESI+) calcd for C₂₉H₂₈F₃N₇O [M+H]⁺: 548.2386, found 548.4866.

### 1.7.24. N-(2-(1H-indazol-6-yl)-1H-benzo[di]imidazol-5-yl)-4-((1-methylpiperidin-4-yl)oxy)-3-(trifluoromethyl)benzamide (8x)

A mixture of N-(2-(1H-indazol-6-yl)-1H-benzo[di]imidazol-5-yl)-4-((1-methylpiperidin-4-yl)oxy)-3-(trifluoromethyl)benzamide was separated in a similar manner to the synthesis of Compound 8c described above to obtain Compound 8x as pure solid in 60.4% yield. m.p. 293-295° C. ¹H NMR (400 MHz, DMSO-d₆) δ 13.56 (s, 1H), 13.40-13.35 (s, 1H), 10.53-10.45 (s, 1H), 8.41 (d, J=7.2 Hz, 1H), 8.36 (d, J=8.5 Hz, 1H), 8.30 (d, J=1.9 Hz, 1H), 8.21-8.15 (s, 1H), 8.12 (s, 1H), 8.00 (d, J=6.1 Hz, 1H), 7.88 (d, J=8.4 Hz, 1H), 7.61 (d, J=8.6 Hz, 1H), 7.56 (dd, J=10.7 Hz, 1H), 7.48 (d, J=10.2 Hz, 1H), 4.17 (dd, J=5.4 Hz, 1H), 3.15 (s, 3H), 2.27 (m, 2H), 2.00 (m, 2H), 1.79 (m, 2H), 1.44 (m, 2H). ¹³C NMR (101 MHz, DMSO-d₆) δ 161.8, 153.5, 153.0, 142.1, 138.1, 137.3, 136.3, 135.8, 134.4, 134.1, 132.1, 131.5, 130.5, 128.3, 127.5, 125.4, 122.1, 117.8, 116.4, 114.9, 114.5, 111.3, 76.3, 53.8, 49.0, 29.5. HRMS (ESI+) calcd for C₂₈H₂₅F₃N₆O₂[M+H]⁺: 535.2069, found 535.4980.

### 1.7.25. N-(2-(1H-indazol-6-yl)-1H-benzo[di]imidazol-5-yl)-3-((4-ethyl-3,3-dimethylpiperazine-1-yl)methyl)-5-(trifluoromethyl)benzamide (8y)

A mixture of N-(2-(1H-indazol-6-yl)-1H-benzo[di]imidazol-5-yl)-3-((4-ethyl-3,3-dimethylpiperazine-1-yl)methyl)-5-(trifluoromethyl)benzamide was separated in a similar manner to the synthesis of Compound 8c described above to obtain Compound 8y as pure solid in 100.0% yield. m.p. 255-257° C. ¹H NMR (400 MHz, DMSO-d₆) δ 13.41 (s, 1H), 13.02 (s, 1H), 10.54-10.48 (s, 1H), 8.35 (s, 1H), 8.28-8.18 (m, 3H), 8.15 (s, 1H), 8.00-7.86 (m, 3H), 7.66 (d, J=8.6 Hz, 1H), 7.50 (d, J=8.3 Hz, 1H), 3.62 (s, 2H), 3.35-3.18 (m, 2H), 2.57 (m, 2H), 2.33 (m, 2H), 2.13 (m, 2H), 1.20 (s, 3H), 0.98 (m, 9H). ¹³C NMR (101 MHz, DMSO-d₆) δ 164.4, 152.4, 141.5, 141.4, 141.2, 137.5, 136.7, 135.6, 134.6, 134.2, 133.8, 132.1, 129.8, 129.5, 128.4, 125.9, 123.2, 121.5, 119.5, 119.1, 116.3, 103.6, 61.1, 54.3, 54.1, 46.3, 43.0, 42.9, 14.6, 14.4. HRMS (ESI+) calcd for C₃₁H₃₂F₃N₇O [M+H]⁺: 576.2699, found 576.5820.

### 1.7.26. N-(2-(1H-indazol-6-yl)-1H-benzo[di]imidazol-5-yl)-3-(4-ethyl-3,3-dimethylpiperazine-1-yl)-5-(trifluoromethyl)benzamide (8z)

A mixture of N-(2-(1H-indazol-6-yl)-1H-benzo[di]imidazol-5-yl)-3-(4-ethyl-3,3-dimethylpiperazine-1-yl)-5-(trifluoromethyl)benzamide was separated in a similar manner to the synthesis of Compound 8c described above to obtain Compound 8z as pure solid in 100.0% yield. m.p. 153-154° C. ¹H NMR (400 MHz, DMSO-d₆) δ 13.41 (s, 1H), 13.03 (s, 1H), 10.43 (s, 1H), 8.35 (s, 1H), 8.27-8.12 (m, 2H), 7.98 (d, J=8.2 Hz, 1H), 7.92 (d, J=8.4 Hz, 1H), 7.75 (s, 1H), 7.66 (s, 1H), 7.52 (m, 2H), 7.40 (s, 1H), 3.35-3.23 (m, 2H), 3.16 (m, 2H), 2.76 (m, 2H), 1.24-1.02 (m, 11H). ¹³C NMR (101 MHz, DMSO-d₆) δ 164.9, 151.6, 141.2, 139.1, 138.1, 137.6, 135.6, 134.6, 134.2, 130.8, 130.5, 128.4, 126.0, 124.0, 123.3, 121.5, 120.6, 119.5, 119.0, 117.9, 114.0, 111.3, 45.6, 34.7, 31.4, 29.0, 25.3, 22.5, 14.4. HRMS (ESI+) calcd for C₃₀H₃₀F₃N₇O [M+H]⁺: 562.6212, found 562.6833.

### Experimental Example 1. Ic₅₀ Measurement and Protein Kinase Profiling

Reaction Biology Corp. Kinase HotSpot SM service (www.reactionbiology.com) was used to determine IC₅₀ values for all compounds and kinase profiles. The analysis protocol was as follows: In a final reaction volume of 25 μL, a peptide substrate, [EAIYAAPFAKKK], 5 μM, ATP 10 μM, and FLT3(h) (5 to 10 mU) were incubated with 25 mM Tris pH 7.5, 0.02 mM EGTA, 0.66 mg/mL myelin basic protein, 10 mM Mg acetate and [γ-33P-ATP] (a concentration of approximately 500 cpm/pmol was required for specific activity). The reaction was initiated by adding the Mg-ATP mixture. After incubation at room temperature for 40 minutes, the reaction was stopped by adding 5 μL of a 3% phosphoric acid solution. Thereafter, 10 μL of the reactant was spotted on a P30 filter mat and washed three times for 5 minutes in 75 mM phosphoric acid and once in methanol before drying and scintillation counting.

### Experimental Example 2. Molecular Modeling

The compound was docked into a FLT3 structure (PDB: 4RT7). Proteins and ligands were prepared with tools from Schrödinger with standard settings, and Glide was used for docking and scoring. A 3D X-ray protein structure of FLT3 wild type in a complex with the ligand was obtained from PDB (code: 4RT7), and prepared using the Protein Preparation Wizard of the Schrödinger Maestro program. All water molecules were removed from the structure and the structure selected as a template. The structure of an inhibitor was generated in a 3D form using Chemdraw and the Schrödinger LigPrep program with OPLS 4 force field. Molecular docking of the compound into the structure of FLT-3 wild type (PDB code: 4RT7) was performed using Schrödinger Glide (version 12.7).

### Experiment Result 1. In Vitro Structure-Activity Relationship (SAR) Study and Structural Modification

All benzimidazole compounds 8a-z were evaluated for the activity for FLT3 and FLT3-D835Y, and the results were shown in Table 1 below. At this time, staurosporine, which exhibited strong inhibitory activity for a non-selectively wide range of protein kinases, was used as a positive control.

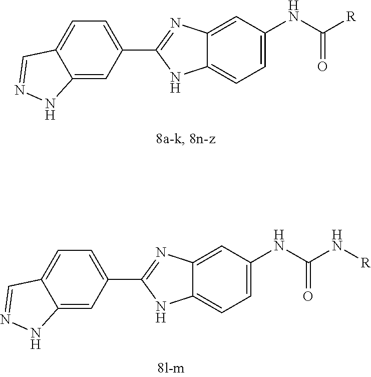

TABLE 1

| | | | FLT3 | | | | FLT3 |
|---|---|---|---|---|---|---|---|
| | | FLT3 | (D835Y) | | | FLT3 | (D835Y) |
| No | R | IC₅₀ (μM) | | No | R | IC₅₀ (μM) | |
| 8a | 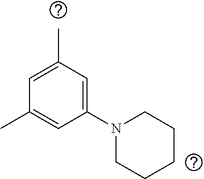 | 0.181 | 0.021 | 8n | 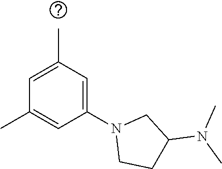 | 0.287 | 0. [symbol] |
| 8b | 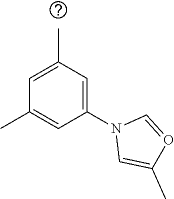 | 0. [symbol] | 0.110 | 8o | 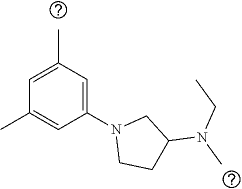 | 0.841 | 0.120 |
| 8c | 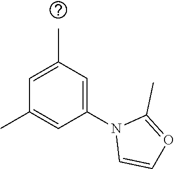 | 0.470 | 6.108 | 8p | 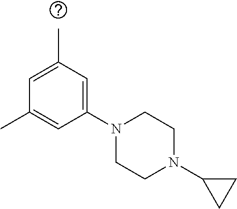 [SMILES: Cc1cc(C)cc(N2CCN(C3CC3)CC2)c1] | 0.409 | 0.131 |
| 8d | 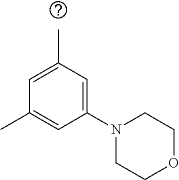 | 1.03 | 0.151 | 8q | 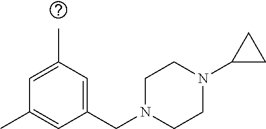 [SMILES: Cc1cc(C)cc(CN2CCN(C3CC3)CC2)c1] | 0.523 | 0.17 [symbol] |
| 8e | 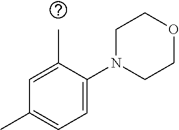 | 0.154 | 0.044 | 8r | 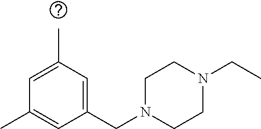 [SMILES: CCN1CCN(Cc2cc(C)cc(C)c2)CC1] | 0.0 [symbol] 6 | 0.606 |
| 8f | 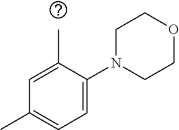 | 2.51 | 0.370 | 8s | 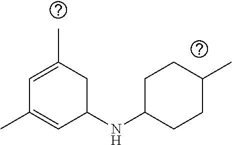 | 0.108 | 0.6 [symbol] |
| 8g | 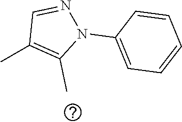 | 1.10 | 0.0 [symbol] | 8t | 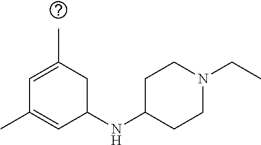 [SMILES: CCN1CCC(NC2C=C(C)C=C(C)C2)CC1] | 0.286 | 0.087 |
| 8h | 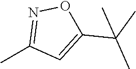 | 0.7 [symbol] | 0.032 | 8u | 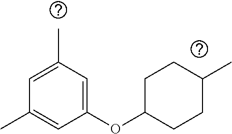 | 0. [symbol] | 0.055 |
| 8i | 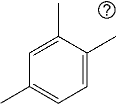 | 0.0 [symbol] | 0. [symbol] | 8v | 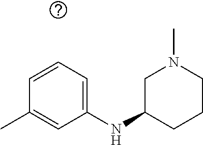 | 0.107 | 0.0 [symbol] 9 |
| 8j | 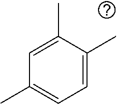 | 0. [symbol] | 0. [symbol] | 8w | 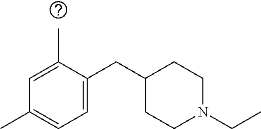 [SMILES: CCN1CCC(Cc2ccc(C)cc2C)CC1] | 0.29 | 0.018 |
| 8k | 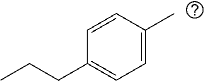 | 0.761 | 0.101 | 8x | 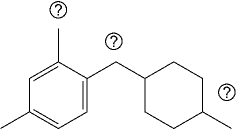 | 0.743 | 0.143 |
| 8l | 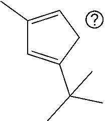 | 1.17 | 0.361 | 8y | 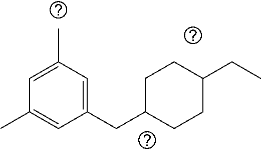 [SMILES: CCC1CCC(Cc2cc(C)cc(C)c2)CC1] | 0.145 | 0.042 |
| 8m | 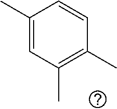 | 3.08 | 1. [symbol] | 8z | 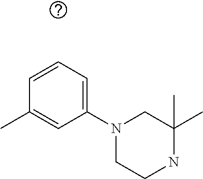 | 0.0 [symbol] | 0.01 [symbol] |

| | | |
|---|---|---|
| Staurosporine | 1.82 nM | 0.073 nM |

[symbol]

indicates data missing or illegible when filed

Most of compounds showed strong and selective activity for FLT3 kinase, mainly stronger activity for FLT3-D835Y. which was associated with secondary resistance mechanisms for FLT3 inhibitors. Compound 8r having an ethyl piperazine moiety showed the most powerful activity, with an IC₅₀ value of 41.6 nM for FLT3 and an IC₅₀ value of 5.64 nM for FLT3-D835Y. Structure-activity relationships (SAR) were inferred from the activity data.

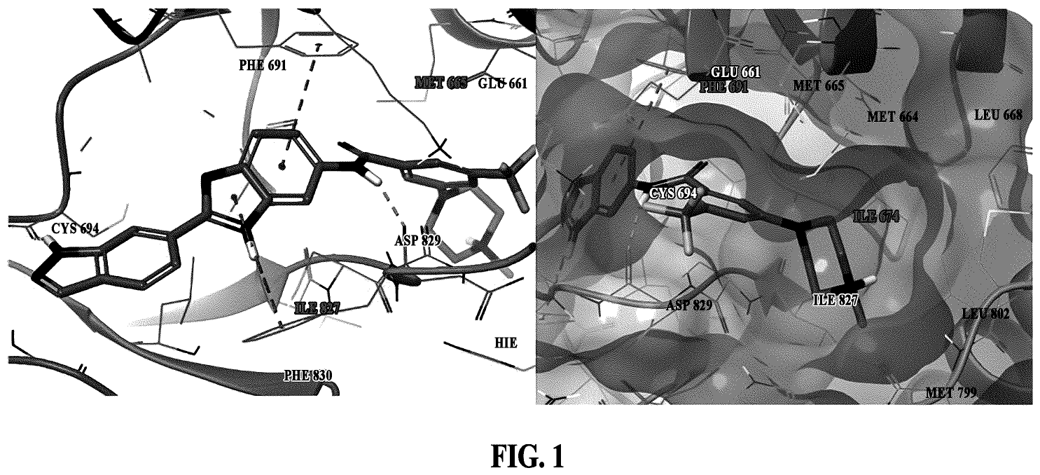

Through investigation of molecular docking, the present inventors confirmed that the indazole structure played a key role as a hinge binder in a type II inhibitor that interacted with a Cys694 residue of FLT3. Accordingly, an indazole moiety was introduced as the hinge binder, and derivatives with a benzimidazole core were synthesized. Most of benzimidazole derivatives retained their activities, and Compounds 8a, 8b, 8d, 8e, 8g, 8h, 8i, and 8k showed particularly improved efficacy on FLT3. In particular, Compounds 8b and 8d showed about 2 to 4 times stronger activity (IC₅₀ values of 0.639 μM and 1.03 μM, respectively) than corresponding quinazoline series thereto (IC₅₀ values of 1.58 μM and 3.98 μM, respectively). Compounds 8a and 8e containing methyl piperazine had the strongest activity among the compounds, and had IC₅₀ values of 0.181 and 0.154 μM for FLT3, respectively. The newly synthesized benzimidazole derivatives of the present disclosure had excellent FLT3 inhibitory activity by binding well to the active site of FLT3 through hydrogen bonding and π-π interaction. NH of the amide group substituted in benzimidazole and NH of indazole formed hydrogen bonds with amide skeletons of Cys694 and Asp829 in FLT3. The fused ring system of benzimidazole interacted with the phenyl side chains of Phe691 and Phe830. Such interaction of the core structures could lead to the development of FLT3 inhibitors with maintained activity and further improved efficacy (FIG. 1).

After the introduction of indazole and benzimidazole, the structure of the derivative, i.e., FLT3 inhibitor, was modified to optimize the activity using a linker connecting a fragment to a pocket adjacent to an ATP binding site. As a result, the pocket increased a space available for the FLT3 inhibitor, and the distal region of the pocket was surrounded by hydrophobic residues such as Met664, Met665, Leu668, I1e674, Met799, Leu802, and Ile827 (FIG. 1).

Compounds 8l and 8m were synthesized to replace the urea linker between the benzimidazole core structure and the phenyl group. Thereafter, the terminal structure was extended by substituting methyl imidazoles 8b and 8c with N,N-dimethyl and diethyl pyrrolidine moieties, respectively. Compound 8n showed three times stronger activity than Compound 8o, which was because the N-methyl group was a substituent having a size suitable to occupy the hydrophobic pocket next to the ATP binding site.

For additional structural optimization, among Compounds 8a-o, a scaffold of Compound 8a, which showed the strongest activity for FLT3, was modified and a methyl substituent was replaced with a cyclopropyl group (8p). Compound 8p maintained excellent inhibitory activity for FLT3. Next, one atom such as C, N, or O was added between a phenyl group and a basic amine substituent to extend the scaffold length and various basic amine substituents 8q-v were added. As a result, Compounds 8r, 8s, and 8v showed improved activity, and specifically, Compounds 8r and 8v showed about 2 to 4 times stronger activity for FLT3. In addition, these compounds showed single nanomolar-unit inhibitory activity for FLT3-D835Y.

Benzimidazole derivatives with 1,3,5-substituted phenyl groups 8r and 8u were about 3 to 7 times stronger than derivatives having 1,3,4-substituted phenyl groups 8w and 8x. 8a and 8e showed only similar activity depending on a substitution direction, and Compounds 8r and 8u showed increased activity compared to 8w and 8x. This means that the 1,3,5-substituted phenyl group occupies a hydrophobic pocket in the binding site of FLT3 kinase.

A dimethyl group was included to optimize the terminals of basic substituents such as piperazine (8y and 8z). Compound 8z had three times stronger activity for FLT3 than 8a (IC₅₀ value of 65.9 nM).

### Experiment Result 2. Enzymatic Inhibition Activity for Wild-Type FLT3 and Activated FLT3 Mutants

For the strongest compound, 8r, the inhibitory activity for FLT3 mutants were further investigated and the results were shown in Table 2 below.

TABLE 2

| Kinase | IC₅₀ |
|---|---|
| FLT3 wild type | 41.6 nM |
| FLT3 (ITD)-NPOS | 41.5 nM |
| FLT3 (ITD)-W51 | 22.8 nM |
| FLT3 (D835Y) | 5.64 nM |
| FLT3 (F594_R595 ins R) | 37.3 nM |
| FLT3 (F594_R595 ins REY) | 41.6 nM |
| FLT3 (R595_E596 ins EY) | 24.8 nM |
| FLT3 (Y591_V592 ins VDFREYEYD) | 49.5 nM |

Compound 8r showed similar or higher levels of efficacy for FLT3-ITD-NPOS and W51 (IC₅₀=41.5 nM, 22.8 nM, respectively) compared to wild-type FLT3 (IC₅₀=41.6 nM). FLT3-ITD mutations mainly occurred in the juxtamembrane domain away from the active site of FLT3, so that FLT3 inhibitors generally showed similar efficacy for FLT3-ITD mutations. However, FLT3-TKD mutations, such as D835Y, occurred in the active site of kinase; point mutations in the site may induce a constitutively active form of FLT3, which was because some type II kinase inhibitors binding to an inactive form of the kinase were generally unable to bind to the FLT3 mutant well enough, and was known as a secondary resistance mechanism of a type II FLT3 inhibitor. However, the benzimidazole derivative of the present disclosure, designed as the type II inhibitor, tended to exhibit stronger activity for FLT3-TKD mutants such as FLT3-D835Y. In particular, Compounds 8r and 8v showed approximately 7 to 12 times stronger activity for FLT3-D835Y (IC₅₀=5.64 nM, 8.86 nM, respectively) than wild-type FLT3 (IC₅₀=41.6 nM, 107 nM, respectively), which suggested that derivatives of the present disclosure may avoid the secondary resistance mechanism. In addition, Compound 8r had similar or stronger activity for other FLT3-TKD mutations, such as FLT3 (F594_R595 ins R), FLT3 (F594_R595 ins REY), FLT3 (R595_E596 ins EY), and FLT3 (Y591_V592 ins VDFREYEYD) compared to wild-type FLT3.

### Experiment Result 3. Protein Kinase Profiling Analysis

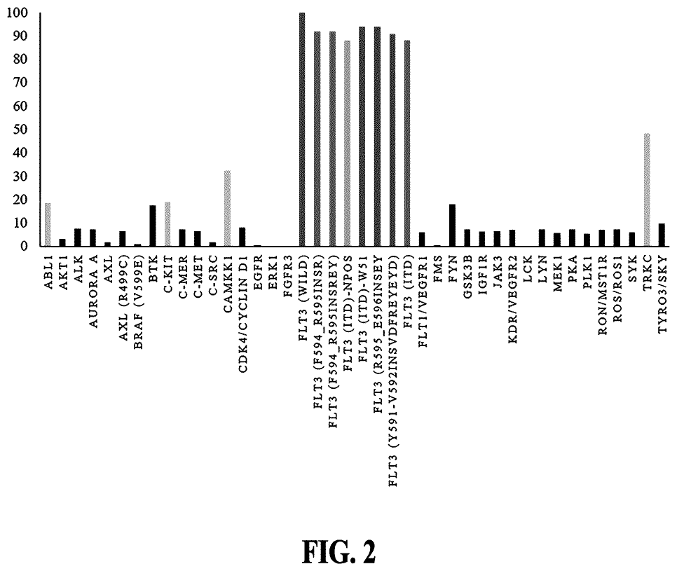

As illustrated in FIG. 2 and Table 3, the inhibitory activity of Compound 8r for 42 different kinases was tested at a single dose concentration of 1 μM.

TABLE 3

| | % | Staurosporine |
|---|---|---|
| Kinase | Inhibition | IC₅₀ (nM) |
| ABL1 | 18.4 | 29.1 |
| AKT1 | 3.09 | 3.21 |
| ALK | 7.50 | 1.76 |
| Aurora A | 7.47 | 2.25 |
| AXL | 1.88 | 6.42 |
| AXL (R499C) | 6.64 | 1.32 |
| BRAF (V599E) | 1.00 | 13.1 |
| BTK | 17.5 | 12.8 |
| c-Kit | 19.0 | 1.05 |
| c-MER | 7.30 | 9.65 |
| c-MET | 6.53 | 26.1 |
| c-Src | 1.94 | 1.59 |
| CAMKK1 | 32.5 | 67.6 |
| CDK4/cyclin D1 | 8.26 | 7.59 |
| EGFR | 0.274 | 39.0 |
| ERK1 | 0 | 15.0 |
| FGFR3 | 0 | 7.63 |
| FLT1/VEGFR1 | 6.00 | 4.32 |
| FLT3 (wild) | 100 | 7.16 |
| FLT3 (F594_R595insR) | 92.0 | 2.34 |
| FLT3 (F594_RS95insREY) | 92.0 | 2.02 |
| FLT3 (ITD)-NPOS | 88.0 | 1.66 |
| FLT3 (ITD)-W51 | 93.9 | 1.95 |
| FLT3 (R595_E596insEY) | 93.9 | 1.72 |
| FLT3 (Y591_ | 90.9 | 2.02 |
| V592insVDFREYEYD) | | |
| FLT3-ITD | 88.0 | 1.86 |
| FMS | 0.531 | 3.10 |
| FYN | 17.96 | <1.00 |
| GSK3b | 7.25 | 8.15 |
| IGF1R | 6.145 | 14.8 |
| JAK3 | 6.42 | <1.00 |
| KDR/VEGFR2 | 7.12 | 11.4 |
| LCK | 0 | 3.56 |
| LYN | 7.40 | <1.00 |
| MEK1 | 5.69 | 56.0 |
| PKA | 7.34 | 1.0 |
| PLK1 | 5.39 | 143 |
| RON/MST1R | 6.99 | 71.2 |
| ROS/ROS1 | 7.40 | <1.00 |
| SYK | 6.01 | <1.00 |
| TRKC | 48.3 | <1.00 |
| TYRO3/SKY | 9.76 | 1.32 |

^{a}Data of GW5074¹
^{b}Data of SCH772984^{2,3}

As a result, it was confirmed that Compound 8r exhibited an excellent selectivity profile. Compound 8r had less than 20% of inhibitory activity for most other kinases, but had stronger inhibitory activity for FLT3-ITD mutants, which were significantly associated with therapeutic targeting in AML. Specifically, Compound 8r had strong inhibitory activity for other FLT3 mutants, including FLT3 (F594_R595insR), FLT3 (F594_R595insREY), FLT3 (ITD)-NPOS, FLT3 (ITD)-W51, FLT3 (R595_E596insEY) and FLT3 (Y591_V592insVDFREYEYD). In addition, the enzymatic inhibitory activity of Compound 8r for ABL1, c-Kit, CAMKK1 and TRKC was evaluated (Table 4).

TABLE 4
IC₅₀

| | ABL1 | c-Kit | CAMKK1 | TRKC |
|---|---|---|---|---|
| 8r | 3.09 μM | 4.57 μM | 0.395 μM | 1.52 μM |

Compound 8r showed excellent activity for wild-type FLT3 and FLT3 mutants (nanomolar IC₅₀ values as shown in Table 2), but did not show excellent efficacy for ABL1 and c-Kit. That is, it was confirmed that the benzimidazole derivative of the present disclosure exhibited selectively high inhibitory activity for wild-type FLT3 and FLT3 mutants.

### Experiment Result 4. Molecular Docking Study

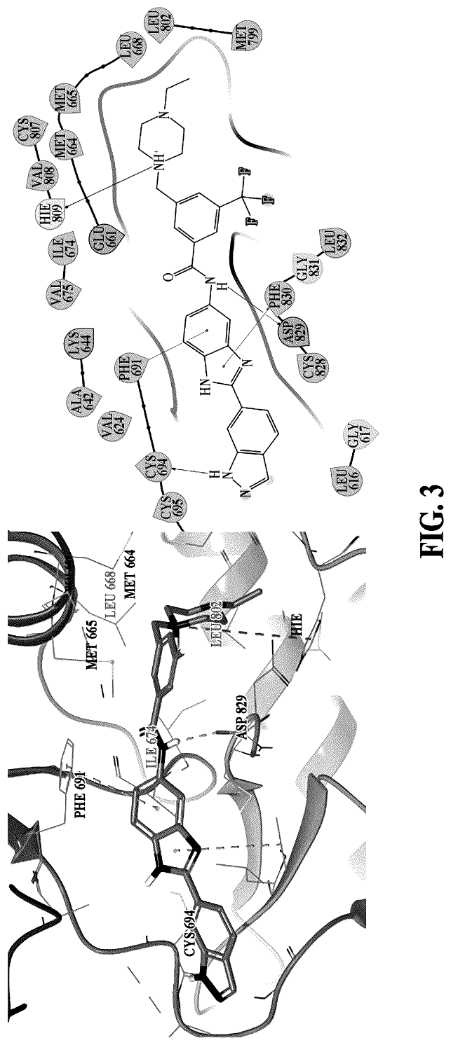

To better understand the interaction between benzimidazole derivatives and FLT3 kinases, the molecular docking of Compound 8r was analyzed using Maestro v12.7 (Scrhodinger Release 2021-1), and the results were shown in FIG. 3. An amino hydrogen atom of 8r indazole formed a hydrogen bond (N—H/O: 1.95 Å) with an amide oxygen atom of Cys694, and another hydrogen bond (1.90 Å) was formed between N—H of a 8r amide bond and an oxygen atom of Asp829. This suggests that the indazole group plays an important role in the receptor-ligand complex. The benzimidazole fused ring of 8r formed t-t interactions with the phenyl groups of Phe691 and Phe830. The interaction also plays an important role in FLT3 kinase inhibition due to a conserved residue Phe691. On the other hand, FMS kinase has the same sequence of Thr663 residue in the active site. Therefore, Compound 8r may not form t-t interaction with Thr663, which exhibits a selective inhibitory activity between FLT3 and FMS kinase. The positively charged nitrogen of the ethyl piperazine ring formed a 7-cation interaction with His809. In addition, the substituted ethyl moiety in piperazine occupies the terminal hydrophobic pocket of the FLT3 active site surrounded by hydrophobic amino acid residues, thereby improving binding affinity and increasing inhibition of kinase activity.

As described above, although the embodiments have been described by the restricted drawings, various modifications and variations may be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result may be achieved.

Therefore, other implementations, other embodiments, and equivalents to the appended claims fall within the scope of the claims to be described below.

### INDUSTRIAL APPLICABILITY

An indazole yl benzimidazole derivative or a pharmaceutically acceptable salt thereof, and the like of the present disclosure exhibits excellent selective inhibitory activity for not only a wild-type FLT3, but also a FLT3 internal tandem duplication (FLT3-ITD) mutation, which is associated with a poor prognosis of acute myeloid leukemia (AML), or FLT3 point mutations, which are frequently observed in AML patients or considered to be a part of a drug resistance mechanism in AML, when administered to a subject. Therefore, the present disclosure may be used for preventing, relieving, or treating cancers including leukemia, inflammatory diseases including arthritis and the like, or osteoporosis.

1. An indazole yl benzimidazole derivative represented by Chemical Formula 1 below or a pharmaceutically acceptable salt thereof: 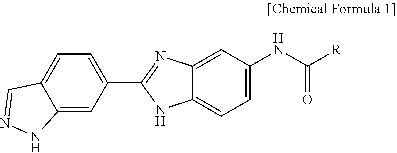
In Chemical Formula 1,
a R group is any one selected from the group consisting of a C₃-C₁₀ aryl group, a C₃-C₁₀ heteroaryl group, a C₅-C₁₀ arylvinyl group, a C₅-C₁₀ heteroarylvinyl group, a C₃-C₁₀ arylamino group, and a C₃-C₁₀ heteroarylamino group,
the R group is substituted with at least one selected from the group consisting of a halogen group, a trifluoromethyl group (CF₃), a C₁-C₆ acyclic or cyclic alkyl group, a C₁-C₆ alkoxy group, a C₃-C₁₀ heterocyclo group, a C₃-C₁₀ aryl group, a C₃-C₁₀ heteroaryl group, a C₃-C₁₀ heterocycloalkyl group, a C₃-C₁₀ heterocycloamino group, and a C₃-C₁₀ heterocycloether group, wherein one or more hydrogens of the heterocyclo group, aryl group, heteroaryl group, heterocycloalkyl group, heterocycloamino group, or heterocycloether group may be each unsubstituted or substituted with at least one selected from the group consisting of a halogen group, a hydroxy group, a cyano group, a nitro group, a C₁-C₆ acyclic or cyclic alkyl group, and a C₁-C₆ alkoxy group.

2. The indazole yl benzimidazole derivative or the pharmaceutically acceptable salt thereof of claim 1, wherein the R group is any one selected from the group consisting of a phenyl group, a pyrazole group, an isoxazole group, a phenylvinyl group, an isoxazole amino group, and a phenylamino group.

3. The indazole yl benzimidazole derivative or the pharmaceutically acceptable salt thereof of claim 1, wherein the R group is substituted with at least one selected from the group consisting of a halogen group, a trifluoromethyl group (CF₃), a tert-butyl group, a methoxy group, a piperazinyl group, a morpholinyl group, a pyrrolidinyl group, a phenyl group, an imidazole group, a piperazinyl methyl group, a piperazinylamino group, a piperidinylamino group, and a piperidinyl ether group, wherein one or more hydrogens of the piperazinyl group, morpholinyl group, pyrrolidinyl group, phenyl group, imidazole group, piperazinylmethyl group, piperazinylamino group, piperidinyl amino group, or piperidinyl ether group may be each unsubstituted or substituted with at least one selected from the group consisting of a halogen group, a hydroxy group, a cyano group, a nitro group, a C₁-C₆ acyclic or cyclic alkyl group, and a C₁-C₆ alkoxy group.

4. The indazole yl benzimidazole derivative or the pharmaceutically acceptable salt thereof of claim 1, wherein the indazole yl benzimidazole derivative represented by Chemical Formula 1 is at least one selected from the group consisting of compounds represented by the following Chemical Formulas:

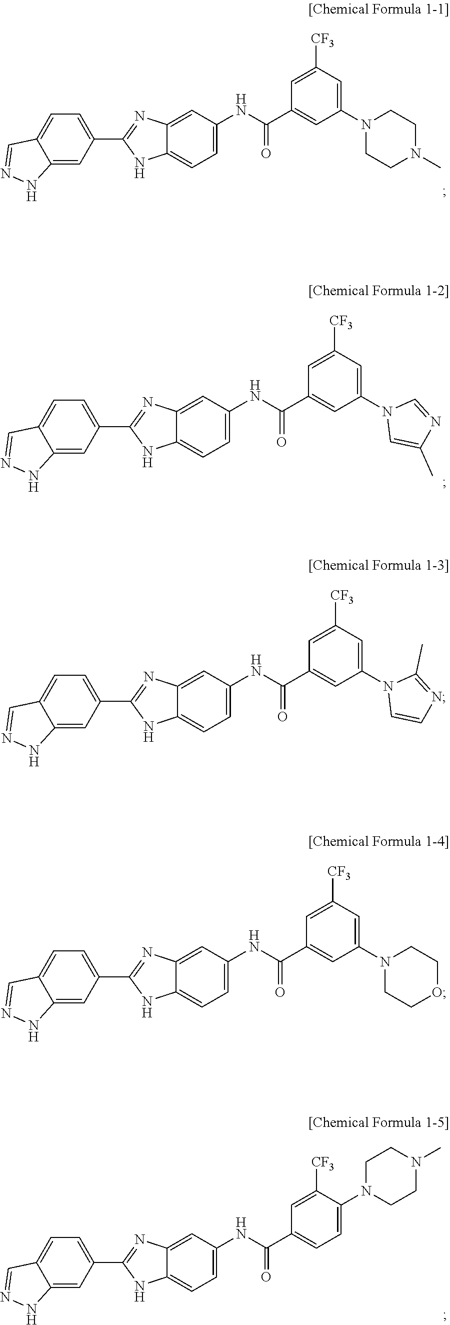 [SMILES: CN1CCN(c2cc(C(=O)Nc3ccc4[nH]c(-c5ccc6cn[nH]c6c5)nc4c3)cc(C(F)(F)F)c2)CC1 ; Cc1cn(-c2cc(C(=O)Nc3ccc4[nH]c(-c5ccc6cn[nH]c6c5)nc4c3)cc(C(F)(F)F)c2)cn1 ; Cc1nccn1-c1cc(C(=O)Nc2ccc3[nH]c(-c4ccc5cn[nH]c5c4)nc3c2)cc(C(F)(F)F)c1 ; O=C(Nc1ccc2[nH]c(-c3ccc4cn[nH]c4c3)nc2c1)c1cc(N2CCOCC2)cc(C(F)(F)F)c1 ; CN1CCN(c2ccc(C(=O)Nc3ccc4[nH]c(-c5ccc6cn[nH]c6c5)nc4c3)cc2C(F)(F)F)CC1]

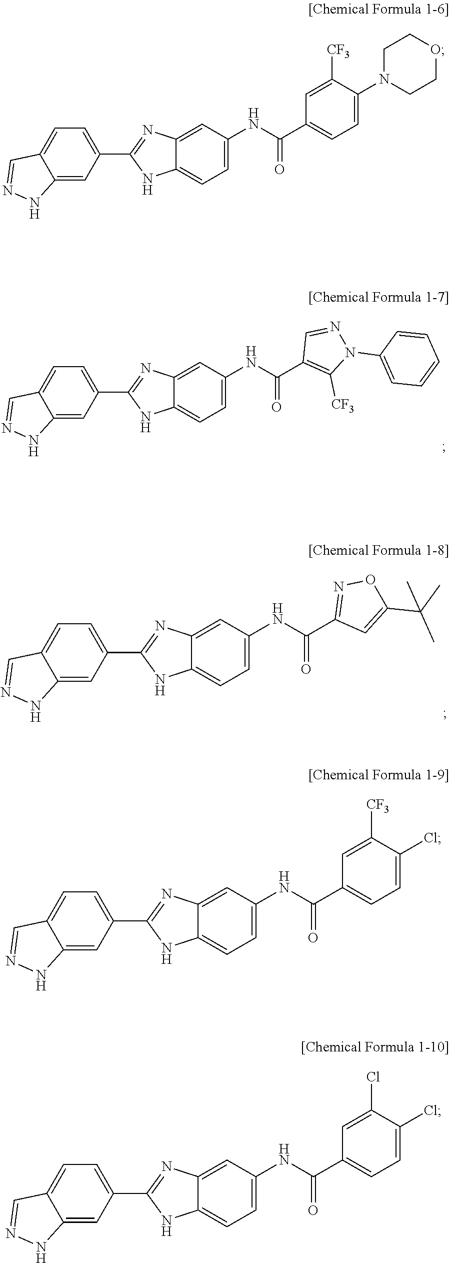 [SMILES: O=C(Nc1ccc2[nH]c(-c3ccc4cn[nH]c4c3)nc2c1)c1ccc(N2CCOCC2)c(C(F)(F)F)c1 ; O=C(Nc1ccc2[nH]c(-c3ccc4cn[nH]c4c3)nc2c1)c1cnn(-c2ccccc2)c1C(F)(F)F ; CC(C)(C)c1cc(C(=O)Nc2ccc3[nH]c(-c4ccc5cn[nH]c5c4)nc3c2)no1 ; O=C(Nc1ccc2[nH]c(-c3ccc4cn[nH]c4c3)nc2c1)c1ccc(Cl)c(C(F)(F)F)c1 ; O=C(Nc1ccc2[nH]c(-c3ccc4cn[nH]c4c3)nc2c1)c1ccc(Cl)c(Cl)c1]

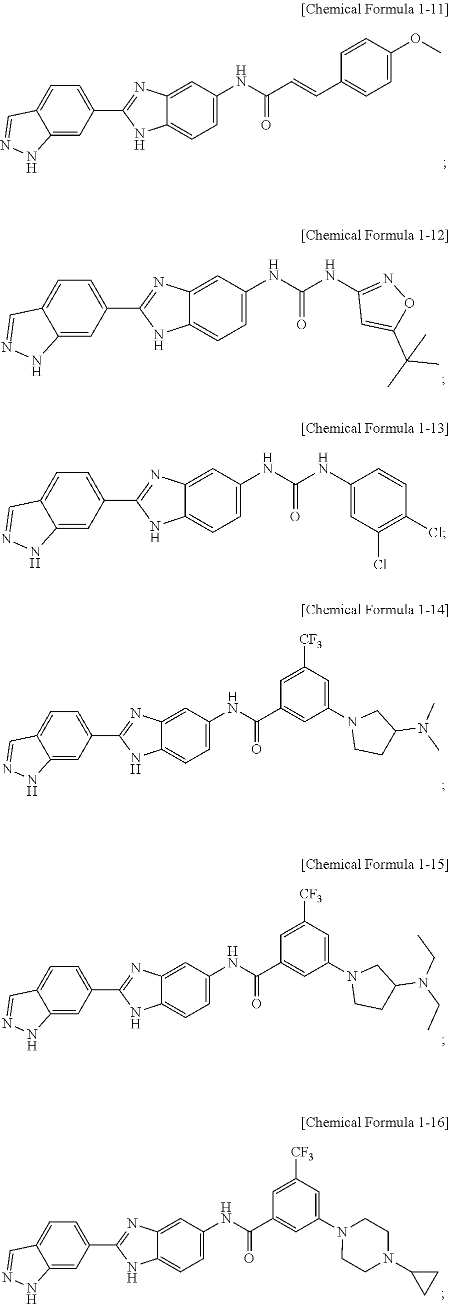 [SMILES: COc1ccc(/C=C/C(=O)Nc2ccc3[nH]c(-c4ccc5cn[nH]c5c4)nc3c2)cc1 ; CC(C)(C)c1cc(NC(=O)Nc2ccc3[nH]c(-c4ccc5cn[nH]c5c4)nc3c2)no1 ; O=C(Nc1ccc(Cl)c(Cl)c1)Nc1ccc2[nH]c(-c3ccc4cn[nH]c4c3)nc2c1 ; CN(C)C1CCN(c2cc(C(=O)Nc3ccc4[nH]c(-c5ccc6cn[nH]c6c5)nc4c3)cc(C(F)(F)F)c2)C1 ; CCN(CC)C1CCN(c2cc(C(=O)Nc3ccc4[nH]c(-c5ccc6cn[nH]c6c5)nc4c3)cc(C(F)(F)F)c2)C1 ; O=C(Nc1ccc2[nH]c(-c3ccc4cn[nH]c4c3)nc2c1)c1cc(N2CCN(C3CC3)CC2)cc(C(F)(F)F)c1]

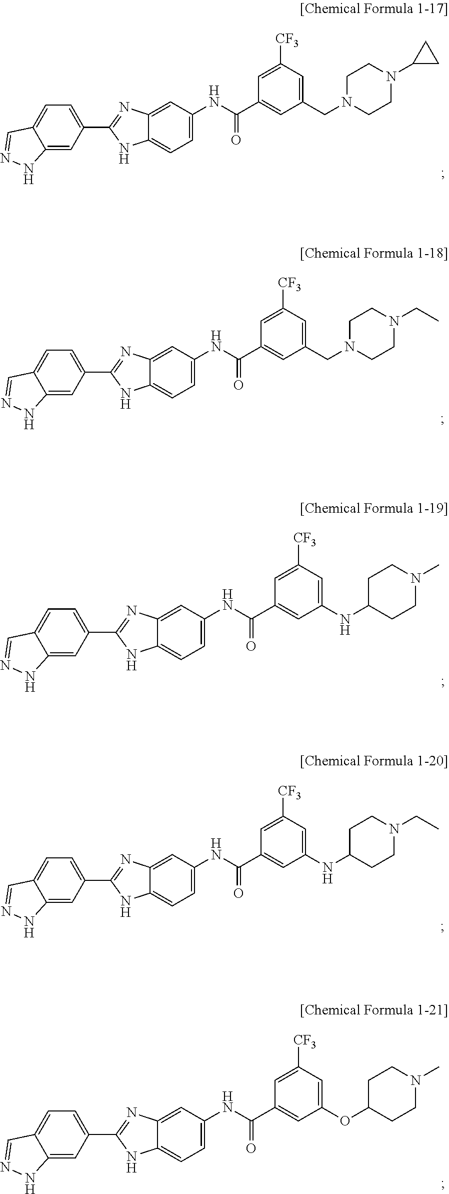 [SMILES: O=C(Nc1ccc2[nH]c(-c3ccc4cn[nH]c4c3)nc2c1)c1cc(CN2CCN(C3CC3)CC2)cc(C(F)(F)F)c1 ; CCN1CCN(Cc2cc(C(=O)Nc3ccc4[nH]c(-c5ccc6cn[nH]c6c5)nc4c3)cc(C(F)(F)F)c2)CC1 ; CN1CCC(Nc2cc(C(=O)Nc3ccc4[nH]c(-c5ccc6cn[nH]c6c5)nc4c3)cc(C(F)(F)F)c2)CC1 ; CCN1CCC(Nc2cc(C(=O)Nc3ccc4[nH]c(-c5ccc6cn[nH]c6c5)nc4c3)cc(C(F)(F)F)c2)CC1 ; CN1CCC(Oc2cc(C(=O)Nc3ccc4[nH]c(-c5ccc6cn[nH]c6c5)nc4c3)cc(C(F)(F)F)c2)CC1]

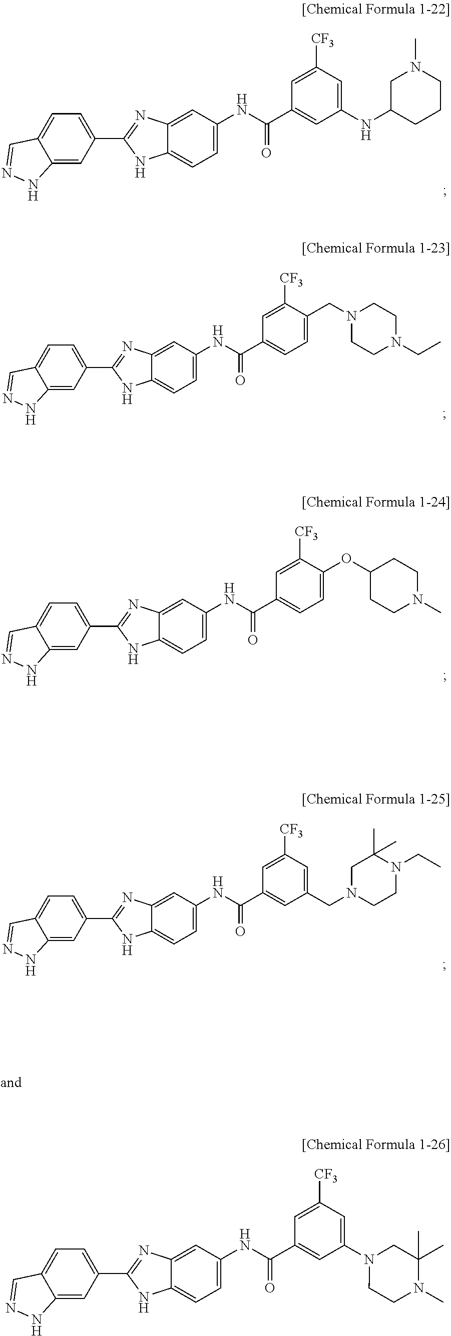 [SMILES: CN1CCCC(Nc2cc(C(=O)Nc3ccc4[nH]c(-c5ccc6cn[nH]c6c5)nc4c3)cc(C(F)(F)F)c2)C1 ; CCN1CCN(Cc2ccc(C(=O)Nc3ccc4[nH]c(-c5ccc6cn[nH]c6c5)nc4c3)cc2C(F)(F)F)CC1 ; CN1CCC(Oc2ccc(C(=O)Nc3ccc4[nH]c(-c5ccc6cn[nH]c6c5)nc4c3)cc2C(F)(F)F)CC1 ; CCN1CCN(Cc2cc(C(=O)Nc3ccc4[nH]c(-c5ccc6cn[nH]c6c5)nc4c3)cc(C(F)(F)F)c2)CC1(C)C ; CN1CCN(c2cc(C(=O)Nc3ccc4[nH]c(-c5ccc6cn[nH]c6c5)nc4c3)cc(C(F)(F)F)c2)CC1(C)C]

5. (canceled)

6. (canceled)

7. A pharmaceutical composition for preventing or treating cancers, inflammatory diseases, or osteoporosis comprising an indazole yl benzimidazole derivative represented by Chemical Formula 1 below or a pharmaceutically acceptable salt thereof as an active ingredient. 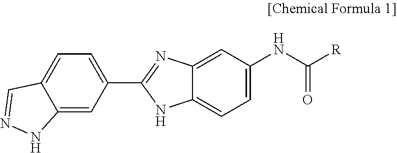
In Chemical Formula 1,
a R group is any one selected from the group consisting of a C₃-C₁₀ aryl group, a C₃-C₁₀ heteroaryl group, a C₅-C₁₀ arylvinyl group, a C₅-C₁₀ heteroarylvinyl group, a C₃-C₁₀ arylamino group, and a C₃-C₁₀ heteroarylamino group,
the R group is substituted with at least one selected from the group consisting of a halogen group, a trifluoromethyl group (CF₃), a C₁-C₆ acyclic or cyclic alkyl group, a C₁-C₆ alkoxy group, a C₃-C₁₀ heterocyclo group, a C₃-C₁₀ aryl group, a C₃-C₁₀ heteroaryl group, a C₃-C₁₀ heterocycloalkyl group, a C₃-C₁₀ heterocycloamino group, and a C₃-C₁₀ heterocycloether group, wherein one or more hydrogens of the heterocyclo group, aryl group, heteroaryl group, heterocycloalkyl group, heterocycloamino group, or heterocycloether group may be each unsubstituted or substituted with at least one selected from the group consisting of a halogen group, a hydroxy group, a cyano group, a nitro group, a C₁-C₆ acyclic or cyclic alkyl group, and a C₁-C₆ alkoxy group.

8. The pharmaceutical composition of claim 7, wherein the pharmaceutical composition further comprises the indazole yl benzimidazole derivative or the pharmaceutically acceptable salt thereof; and one or more additional ingredients selected from the group consisting of pharmaceutically acceptable carriers, excipients, diluents, stabilizers and preservatives.

9. The pharmaceutical composition of claim 7, wherein the pharmaceutical composition has formulations selected from the group consisting of powders, granules, tablets, capsules, injections, and mixtures thereof.

10. The pharmaceutical composition of claim 7, wherein the cancer is at least one selected from the group consisting of leukemia, lymphoma, osteosarcoma, skin cancer, breast cancer, uterine cancer, esophageal cancer, stomach cancer, brain tumor, colon cancer, rectal cancer, colorectal cancer, lung cancer, ovarian cancer, cervical cancer, endometrial cancer, vulvar cancer, kidney cancer, blood cancer, pancreatic cancer, prostate cancer, testicular cancer, laryngeal cancer, head and neck cancer, thyroid cancer, liver cancer, bladder cancer, thymus cancer, urethral cancer, and bronchial cancer.

11. The pharmaceutical composition of claim 10, wherein the leukemia is at least one selected from the group consisting of acute myelogenous leukemia (AML), chronic myelogenous leukemia (CML), acute lymphocytic leukemia (ALL), chronic lymphocytic leukemia (CLL), acute promyelocytic leukemia (APL), hairy cell leukemia, and chronic neutrophilic leukemia (CNL).

12. The pharmaceutical composition of claim 7, wherein the inflammatory disease is at least one selected from the group consisting of arthritis, osteoarthritis, rheumatoid arthritis, psoriatic arthritis, inflammatory arthritis, polyarthritis, glomerulonephritis, inflammatory bowel disease, polymyositis, atopic dermatitis, allergic rhinitis, and asthma.

13. A method for preventing or treating cancers, inflammatory diseases, or osteoporosis comprising administering an indazole yl benzimidazole derivative represented by Chemical Formula 1 below or a pharmaceutically acceptable salt thereof to a subject. 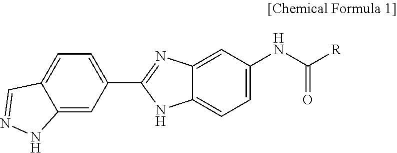
In Chemical Formula 1,
a R group is any one selected from the group consisting of a C₃-C₁₀ aryl group, a C₃-C₁₀ heteroaryl group, a C₅-C₁₀ arylvinyl group, a C₅-C₁₀ heteroarylvinyl group, a C₃-C₁₀ arylamino group, and a C₃-C₁₀ heteroarylamino group,
the R group is substituted with at least one selected from the group consisting of a halogen group, a trifluoromethyl group (CF₃), a C₁-C₆ acyclic or cyclic alkyl group, a C₁-C₆ alkoxy group, C₃-C₁₀ heterocyclo group, a C₃-C₁₀ aryl group, a C₃-C₁₀ heteroaryl group, a C₃-C₁₀ heterocycloalkyl group, a C₃-C₁₀ heterocycloamino group, and a C₃-C₁₀ heterocycloether group, wherein one or more hydrogens of the heterocyclo group, aryl group, heteroaryl group, heterocycloalkyl group, heterocycloamino group, or heterocycloether group may be each unsubstituted or substituted with at least one selected from the group consisting of a halogen group, a hydroxy group, a cyano group, a nitro group, a C₁-C₆ acyclic or cyclic alkyl group, and a C₁-C₆ alkoxy group.

14. The method for preventing or treating cancers, inflammatory diseases, or osteoporosis of claim 13, wherein the cancer is at least one selected from the group consisting of leukemia, lymphoma, osteosarcoma, skin cancer, breast cancer, uterine cancer, esophageal cancer, stomach cancer, brain tumor, colon cancer, rectal cancer, colorectal cancer, lung cancer, ovarian cancer, cervical cancer, endometrial cancer, vulvar cancer, kidney cancer, blood cancer, pancreatic cancer, prostate cancer, testicular cancer, laryngeal cancer, head and neck cancer, thyroid cancer, liver cancer, bladder cancer, thymus cancer, urethral cancer, and bronchial cancer.

15. (canceled)
